(19) United States
(12) Patent Application Publication
(10) Pub. No.: US 2026/0354746 A1
(43) Pub. Date: Nov. 12, 2026

(54) ANALYSIS SYSTEM AND PROBE ASSEMBLY

(71) Applicant: LUMINOMA DIAGNOSTICS LIMITED, Auckland (NZ)

(72) Inventors: Michel Karin NIEUWOUDT, Auckland (NZ); Hannah Marie MATTHEWS, Auckland (NZ); Hannah Elizabeth SHERIDAN, Auckland (NZ); Samuel Marc LEVESTAM, Auckland (NZ); Marco BONESI, Auckland (NZ); Alex Michael MARGIOTT, Newark, CA (US); Robert John de Saint PHALLE, Newark, CA (US); Todd Louis HARRIS, Newark, CA (US); Mark Gustav LEVATICH, Newark, CA (US); Russell Biddle FORD, Newark, CA (US); Tobias FUNK, Newark, CA (US); Christopher Eugene TODD, Newark, CA (US)

(73) Assignee: LUMINOMA DIAGNOSTICS LIMITED, Auckland (NZ)

(21) Appl. No.: 19/488,471

(22) PCT Filed: May 31, 2024
(86) PCT No.: PCT/NZ2024/050063

§ 371 Date: Nov. 26, 2025

(30) Foreign Application Priority Data
May 31, 2023 (AU) 2023901702

(51) Int. Cl. A61B 5/00

(52) U.S. Cl. CPC A61B 5/444; A61B 5/0075; A61B 5/0077; A61B 5/6843; A61B 5/7425

(57) ABSTRACT

Forms of the technology relate to a probe assembly for spectral analysis of a subject, particularly a skin region. The probe assembly comprises a housing configured to retain a probe for Raman spectroscopy inside the housing. A probe tip shield has a distal end defining an opening to expose a tip of the probe to the skin region. The probe tip shield shields the tip from ambient light at least at wavelengths of sensitivity for Raman spectroscopy. A shape of the probe tip shield is able to be adapted to conform to a region of the subject's body. A retraction mechanism may control a pressure exerted by the tip on the skin. A camera may be positioned inside the housing to capture visual images of the skin.

### 1. FIELD OF THE TECHNOLOGY

Forms of the technology are directed to analysis systems, devices and methods. Forms of the technology are directed towards a probe assembly for an analysis system. The systems, devices and methods may particularly be applied to the analysis of a patient's skin, for example to analyse skin lesions, and to diagnose certain medical conditions.

### 2. BACKGROUND TO THE TECHNOLOGY

Skin cancer is the most common form of cancer worldwide, globally accounting for at least 40% of cancer cases, and its incidence is steadily increasing. Early detection and accurate diagnosis are crucial for effective treatment and improved patient outcomes.

Conventionally, the diagnosis of skin cancer involves a visual examination of suspicious skin lesions by dermatologists, followed by a biopsy and histopathological examination. This process may be subjective, time-consuming, invasive, and often requires specialised facilities and trained personnel.

In recent years, there has been a growing interest in the development of non-invasive diagnostic techniques to improve the efficiency and accuracy of skin cancer diagnosis. Spectroscopic and imaging techniques, such as Raman spectroscopy, fluorescence spectroscopy, terahertz spectroscopy and optical coherence tomography, have shown great potential in this regard.

Raman spectroscopy is a non-destructive analytical technique that provides information about the molecular composition of a sample by measuring the inelastic scattering of photons. Several research studies have demonstrated the utility of Raman spectroscopy for skin cancer diagnosis. By analysing the Raman spectra of skin tissue samples, various molecular changes associated with different types of skin cancer can be identified. These changes include alterations in protein content, lipid composition, nucleic acids, and other biomolecules. The ability to detect and quantify these molecular changes provides a valuable tool for the diagnosis and classification of skin cancer.

Existing devices for skin cancer diagnosis based on different spectroscopic techniques are limited in their capabilities and practicality. These devices often require complex calibration procedures, bulky equipment, and extensive data analysis, which restrict their use to laboratory settings or specialised medical facilities. Additionally, the methodologies on which these different forms of spectroscopy are based in these devices, may result in them lacking the necessary sensitivity and specificity required for accurate diagnosis in a diverse range of patient populations.

It may be useful to identify skin lesions irrespective of whether they are cancerous. Knowing the type of skin lesion may assist in conferring a benign diagnosis, and may also be useful for determining suitable treatments for non-cancerous lesions.

### 3. OBJECT OF THE TECHNOLOGY

It is an object of the technology to provide an improved system, device and/or method for analysing a subject. Alternatively, it is an object of the technology to provide an improved system for spectral analysis of a subject, which may be a skin lesion. Alternatively, it is an object of the technology to provide an improved probe assembly for spectral analysis of a system, for example a probe assembly that may be used as part of a spectral analysis system. Alternatively, it is an object of the technology to at least provide the public with a useful choice.

### 4. SUMMARY OF THE TECHNOLOGY

According to aspects of the technology there are provided systems, devices and methods for analysing a subject, for example a patient's skin. In certain forms, the systems, devices and methods may use Raman spectroscopy techniques to analyse the subject. The analysis may be used to identify skin lesions and/or to diagnose certain conditions and/or diseases, for example skin cancers.

According to one aspect of the technology there is provided a system for analysing a subject comprising a probe, a stimulus generator, a detector and a processor. Parts of the system, for example the detector, may be considered to be a spectrometer. In certain forms, the probe is configured to illuminate the subject with electromagnetic radiation. The probe may be further configured to receive electromagnetic radiation from the subject. The probe may be further configured to convey the received electromagnetic radiation to the detector. The detector may be configured to generate a signal indicative of the received electromagnetic radiation and convey the signal to the processor. In certain forms, the processor is configured to receive the signal indicative of the received electromagnetic radiation. The processor may be further configured to perform spectral analysis on the signal and generate an indication of skin health, for example to analyse a skin region of the subject and to identify a skin lesion in the skin region.

According to one aspect, there is provided a probe assembly for use in a spectrometer, which may be used for analysing a subject, for example a region of a patient's skin. The probe assembly may comprise a probe. In some forms, the probe assembly may be configured to be handheld.

In some forms, the probe assembly may be configured to substantially shield the subject from ambient light when the probe is in use. For example, the probe assembly may comprise a probe tip shield configured to shield the tip from ambient light, for example from ambient light at least at wavelengths of sensitivity for Raman spectroscopy. The probe assembly may be configured such that a shape of the distal end of the probe tip shield is able to be adapted to conform to a region of the subject's body surrounding the skin region.

In some forms, the probe assembly may comprise a probe housing and the probe may be housed within the probe housing. Furthermore, the probe may be retractable relative to the probe housing.

In some forms, the probe assembly may comprise a retraction mechanism for moving the probe between an extended position and a retracted position. The retraction mechanism may comprise an actuator configured to act on the probe to extend and retract the probe. The retraction mechanism may further comprise a pressure control mechanism for controlling a pressure exerted by the tip on the skin region when the probe is in the extended position.

In some forms, the probe assembly comprises a viewing assembly to enable a user to view the subject that is analysed by the probe in use. The viewing assembly may comprise a camera and a screen configured to display images from the camera.

In some forms, the probe assembly may comprise a camera positioned inside the housing to capture visual images of the skin region when the distal end abuts a region of the subject's body surrounding the skin region.

According to one aspect of the invention there is provided a probe assembly for spectral analysis of a subject. The probe assembly may comprise a housing configured to retain a probe inside the housing. The housing may comprise a housing body and a probe tip shield having a proximal end provided to an end of the housing body, and a distal end defining an opening to expose a tip of the probe to the subject. The probe tip shield may be configured to substantially shield the tip from ambient light.

In certain forms, the probe tip shield and housing body are configured to fully shield the tip of the probe from ambient light when the distal end abuts the subject.

In certain forms, the probe tip shield is configured to substantially shield the tip of the probe from direct ambient light incident on the tip or from ambient light reflected off a surface of the subject. In certain forms, the probe tip shield is configured to substantially shield the tip of the probe from ambient light scattered through the subject.

In certain forms, the probe tip shield comprises a body tapering between the proximal end and the distal end. For example, the probe tip shield may be frustoconical.

In certain forms, the probe tip shield comprises a skirt extending outwardly from the distal end of the probe tip shield to cover a surface region substantially surrounding the subject in use.

In certain forms, the probe assembly comprises a retraction mechanism for moving the probe between an extended position and a retracted position, where, in the extended position, the tip is positioned substantially flush with the distal end of the probe tip shield, and, in the retracted position, the tip is positioned inside the housing.

In certain forms of the technology, the probe assembly comprises a viewing assembly to enable a user to view the subject when the distal end abuts the subject.

In certain forms, the viewing assembly comprises a camera positioned to capture images of the subject. The camera may be positioned inside the housing. The viewing assembly may further comprise a light source configured to illuminate the subject, the light source being positioned inside the housing.

In certain forms, the viewing assembly may also comprise a screen configured to display images from the camera to the user. The screen may be positioned outside the housing, or a wall of the housing may comprise the screen.

According to another aspect of the invention there is provided a probe assembly for spectral analysis of a skin region of a subject. The probe assembly may comprise a housing configured to retain a probe for Raman spectroscopy inside the housing. The housing may comprise a housing body and a probe tip shield having a proximal end provided to an end of the housing body, and a distal end defining an opening to expose a tip of the probe to the skin region. The probe tip shield may be configured to shield the tip from ambient light at least at wavelengths of sensitivity for Raman spectroscopy. The probe assembly may be configured such that a shape of the distal end of the probe tip shield is able to be adapted to conform to a region of the subject's body surrounding the skin region.

In certain forms, the probe tip shield may be configured to be substantially flexible such the distal end is able to conform to the region of the subject's body surrounding the skin region.

In certain forms, the distal end of the probe tip shield may comprise one or more cavities configured to facilitate the distal end conforming to the region of the subject's body surrounding the skin region.

In certain forms, the probe tip shield may comprise one or more folds enabling the probe tip shield to deform in shape to facilitate the distal end conforming to the region of the subject's body surrounding the skin region.

In certain forms, the probe assembly may comprise a plurality of probe tip shields. The distal end of each of the plurality of probe tip shields may be different in shape and/or size. The proximal end of each of the plurality of probe tip shields may be interchangeably attachable to the housing body.

In certain forms, the probe tip shield may taper from a wider distal end to a narrower proximal end.

In certain forms, the probe tip shield may be configured such that the region of the subject's body surrounding the skin region has a minimum width sufficient to substantially eliminate sub-surface scattered ambient light being received by the probe, for example approximately 5 cm.

In certain forms, the probe tip shield may be configured to be substantially opaque to light at substantially all wavelengths.

In certain forms, the probe assembly may further comprise a camera positioned inside the housing to capture visual images of the skin region when the distal end abuts the region of the subject's body surrounding the skin region. The probe assembly may further comprise a light source to illuminate the skin region when the camera captures visual images of the skin region.

According to another aspect of the invention there is provided a probe assembly for spectral analysis of a skin region of a subject. The probe assembly may comprise a housing configured to retain a probe inside the housing. The housing may comprise a housing body and a probe tip shield having a proximal end provided to an end of the housing body, and a distal end defining an opening to expose a tip of the probe to the skin region. The probe assembly may further comprise a retraction mechanism for moving the probe between an extended position and a retracted position. The retraction mechanism may comprise an actuator configured to act on the probe to extend and retract the probe. The retraction mechanism may further comprise a pressure control mechanism for controlling a pressure exerted by the tip on the skin region when the probe is in the extended position.

In certain forms, the pressure control mechanism may comprise an actuator controller to control the actuator to limit movement of the probe in the extended position.

In certain forms, the pressure control mechanism may comprise a pressure sensor configured to sense pressure exerted on the probe. The actuator controller may be configured to limit movement of the probe based on pressure on the probe sensed by the pressure sensor.

In certain forms, the pressure control mechanism may comprise a prod assembly configured to urge a prod member against a region of the subject's body proximate the skin region. The prod member may be positioned inside the probe tip shield.

In certain forms, the pressure control mechanism may comprise an imaging assembly configured to determine when the tip is in contact with the skin region. The actuator controller may be configured to limit movement of the probe based on the determination of the imaging assembly.

In certain forms, the pressure control mechanism may comprise a mapping assembly configured to determine shape of the skin region. The actuator controller may be configured to limit movement of the probe based on the determination of the mapping assembly.

In certain forms, the probe assembly may further comprise a viewing assembly to enable a user to view the skin region when the distal end abuts the skin region. In certain forms, the viewing assembly may comprise a camera positioned inside the housing to capture visual images of the skin region.

In certain forms, the probe tip shield may be configured to shield the tip from ambient light at least at wavelengths of sensitivity for Raman spectroscopy.

In certain forms, the probe assembly may be configured such that a shape of the distal end of the probe tip shield is able to be adapted to conform to the region of the subject's body surrounding the skin region.

According to another aspect of the invention there is provided a probe assembly for spectral analysis of a skin region of a subject. The probe assembly may comprise a housing configured to retain a probe inside the housing. The housing may comprise a housing body and a probe tip shield having a proximal end provided to an end of the housing body, and a distal end defining an opening to expose a tip of the probe to the skin region. The probe assembly may further comprise a camera positioned inside the housing to capture visual images of the skin region when the distal end abuts a region of the subject's body surrounding the skin region.

In certain forms, the probe tip shield may be configured to shield the tip from ambient light at least at wavelengths of sensitivity for Raman spectroscopy. The probe assembly may further comprise a light source to illuminate the skin region when the camera captures visual images of the skin region.

In certain forms, the probe assembly may be configured such that a shape of the distal end of the probe tip shield is able to be adapted to conform to the region of the subject's body surrounding the skin region.

In certain forms, the probe assembly may further comprise a screen configured to display images from the camera to a user.

In certain forms, the probe assembly may be configured to communicate the images from the camera to a location remote from the probe assembly.

According to another aspect of the invention there is provided a probe assembly for spectral analysis of a skin region of a subject. The probe assembly may comprise a housing configured to retain a probe for Raman spectroscopy inside the housing. The housing may comprise a housing body and a probe tip shield having a proximal end provided to an end of the housing body, and a distal end defining an opening to expose a tip of the probe to the skin region. The probe tip shield may be configured to shield the tip from ambient light at least at wavelengths of sensitivity for Raman spectroscopy. The probe tip shield may comprise a skirt extending radially outwardly from the distal end of the probe tip shield to cover a surface region surrounding the skin region in use. The skirt may be substantially opaque at least at wavelengths of sensitivity for Raman spectroscopy.

In certain forms, a radially outer edge of the skirt has a minimum width sufficient to substantially eliminate sub-surface scattered ambient light being received by the probe, for example approximately 5 cm. In certain forms, the skirt may extend radially perpendicularly outwardly from the distal end of the probe tip shield. In certain forms, the skirt may be formed so as to be flexible or semi-rigid so as to be able to flex when pushed up against the subject.

According to another aspect of the invention there is provided a probe assembly for spectral analysis of a skin region of a subject. The probe assembly may comprise a housing configured to retain a probe inside the housing. The housing may comprise a housing body and a probe tip shield having a proximal end provided to an end of the housing body, and a distal end defining an opening to expose a tip of the probe to the skin region. The probe assembly may further comprise a retraction mechanism for moving the probe between an extended position and a retracted position, where, in the extended position, the tip is positioned substantially flush with the distal end of the probe tip shield, and, in the retracted position, the tip is positioned inside the housing. The probe assembly may further comprise a viewing assembly to enable a user to view the skin region when the distal end abuts a region of the subject's body surrounding the skin region.

In certain forms, the viewing assembly may comprise a camera positioned inside the housing to capture visual images of the skin region.

According to another aspect of the invention there is provided a system for spectral analysis of a skin region of a subject. The system may comprise a probe assembly comprising a housing configured to retain a probe inside the housing. The system may further comprise a probe assembly holder for retaining the probe assembly when not in use. The system may further comprise a calibration member positioned relative to the probe assembly holder such that, when the probe assembly is retained in the probe assembly holder, the calibration member is positioned for analysis by the probe. The calibration member may produce a known spectrum when exposed to light from the probe.

In certain forms, the calibration member may produce a known Raman spectrum when exposed to light from the probe.

In certain forms, the system may further comprise a power meter configured to measure a power of light emitted by the probe. The power meter may comprise a receiver positioned relative to the probe assembly holder such that, when the probe assembly is retained in the probe assembly holder, the receiver is positioned to receive light emitted by the probe.

In certain forms, the system may further comprise a calibration mechanism configured to alter the relative positions of the calibration member and the receiver relative to the probe assembly holder such that, in a first configuration, the calibration member is positioned in front of the probe when the probe is retained in the probe assembly holder and, in a second configuration, the receiver is positioned in front of the probe when the probe is retained in the probe assembly holder.

According to one aspect of the invention there is provided a probe assembly for spectral analysis of a subject. The probe assembly may comprise a housing configured to retain a probe inside the housing.

The probe assembly may further comprise a calibration member positioned inside the housing. The calibration member may produce a known spectrum when exposed to light from the probe. The probe assembly may further comprise a calibration mechanism configured to alter the relative position of the calibration member and the probe between a calibration configuration and a subject configuration so that, in the calibration configuration, the probe is positioned to analyse the calibration member, and, in the subject configuration, the probe is positioned to analyse the subject.

In certain forms, the calibration mechanism may be configured to move the calibration member such that, in the calibration configuration, the calibration member is positioned directly in front of the probe, and, in the subject configuration, the calibration member is not positioned directly in front of the probe.

In certain forms, the probe assembly may comprise a retraction mechanism configured to move the probe between an extended position and a retracted position, where, in the extended position, the tip is positioned substantially flush with an end of the housing, and, in the retracted position, the tip is positioned inside the housing. In certain forms, in the calibration configuration, the probe may be in the retracted position, and, in the subject configuration, the probe may be in the extended position.

In certain forms, the housing may be configured such that the probe assembly is suitable for holding in a user's hand during use.

In certain forms, the probe assembly may further comprise the probe. In certain forms, the probe may comprise one or more illuminating light guides configured to convey light to illuminate the subject. The probe may further comprise one or more collecting light guides configured to collect light from the subject and convey the collected light along the one or more collecting light guides.

According to another aspect of the invention there is provided a probe assembly for spectral analysis of a subject. The probe assembly may comprise a housing configured to retain a probe inside the housing. The housing may comprise a housing body and a probe tip shield having a proximal end provided to an end of the housing body, and a distal end defining an opening to expose a tip of the probe to the skin region. The probe assembly may further comprise a retraction mechanism for moving the probe between an extended position and a retracted position, where, in the retracted position, the tip is positioned inside the housing. The probe assembly may further comprise an ultra-violet light source positioned inside the housing and configured to illuminate the tip with ultra-violet light when the probe is in the retracted position.

According to another aspect of the technology, there is provided a system for spectral analysis of a subject. The system may comprise a probe assembly according to another aspect of the technology. The system may further comprise a stimulus generator for generating light for exposure of the subject. The stimulus generator may provide light to the one or more illuminating light guides of the probe assembly. The system may further comprise a detector for receiving collected light from the subject, for example for receiving collected light from the one or more collecting light guides. The detector may generate a signal indicative of the collected light from the subject. The system may further comprise a processor for analysing the signal indicative of the collected light from the subject. The processor may be configured to output information indicative of the result of the analysis.

In certain forms, the system may comprise a portable unit housing the stimulus generator, the detector and the processor. In some forms, the probe assembly, the one or more illuminating light guides and the one or more collecting light guides may also be housed in the portable unit.

Further aspects of the technology, which should be considered in all its novel aspects, will become apparent to those skilled in the art upon reading of the following description which provides at least one example of a practical application of the technology.

### 5. BRIEF DESCRIPTION OF THE DRAWINGS

One or more embodiments of the technology will be described below by way of example only, and without intending to be limiting, with reference to the following drawings, in which:

FIG. 1 is a perspective view illustration of an exemplary analysis system 100 for analysing a subject according to one form of the technology;

FIG. 2 is a schematic illustration of an exemplary analysis system 100 for analysing a subject according to certain forms of the technology;

FIG. 3 is a perspective view illustration of an exemplary probe assembly 200 for analysing a subject according to one form of the technology;

FIG. 4 is a cross-sectional view illustration of an exemplary probe assembly 200 for analysing a subject according to one form of the technology;

FIG. 5 is a cross-sectional view illustration of an exemplary probe assembly 200 for analysing a subject according to another form of the technology;

FIG. 6 is a perspective view illustration of an exemplary probe tip shield 7 and skirt 6 according to one form of the technology;

FIG. 7 is a perspective view illustration of an exemplary probe tip shield 7 and skirt 6 according to another form of the technology;

FIG. 8 is an end view illustration of an end wall 31 of the probe assembly 200 of FIG. 4;

FIG. 9 is an illustration of an inside of the end wall 31 of the probe assembly 200 of FIG. 4;

FIG. 10 is an exploded perspective view illustration of a tip 5 of a probe 4 according to one form of the technology;

FIG. 11 is a cross-sectional side view illustration of a tip 5 of a probe 4 according to another form of the technology;

FIGS. 12A-C are plan view illustrations of skin regions of a subject being spectrally analysed and a region of the subject's body surrounding the skin region;

FIG. 13A is an illustration of an exemplary probe assembly 200 according to another form of the technology being used on a patient;

FIGS. 13B-C are cross-sectional view illustrations of the exemplary probe assembly 200 of FIG. 13A;

FIG. 14A is an illustration of an exemplary probe assembly 200 according to another form of the technology being used on a patient;

FIGS. 14B-C are cross-sectional view illustrations of the exemplary probe assembly 200 of FIG. 14A;

FIGS. 15A-B are illustrations of an exemplary probe assembly 200 according to another form of the technology;

FIG. 16 is an illustration of an exemplary probe assembly 200 according to another form of the technology being used on a patient;

FIG. 17A is a cross-sectional view illustration of an exemplary probe assembly 200 according to another form of the technology;

FIG. 17B is a schematic plan view illustration of a skin region of a subject being spectrally analysed using the exemplary probe assembly 200 of FIG. 17A;

FIG. 18A is a cross-sectional view illustration of an exemplary probe assembly 200 according to another form of the technology;

FIG. 18B is a schematic plan view illustration of a skin region of a subject being spectrally analysed using the exemplary probe assembly 200 of FIG. 18A;

FIGS. 19A-B are cross-sectional view illustrations of an exemplary probe assembly 200 comprising a UV sterilization mechanism for sterilizing a probe tip according to another form of the technology;

FIG. 20 is a cross-sectional view illustration of an exemplary probe assembly 200 according to another form of the technology;

FIG. 21 is a schematic illustration of an exemplary analysis system 100 for analysing a subject according to certain forms of the technology;

FIGS. 22A-C are cross-sectional view illustrations of a calibration mechanism of an exemplary analysis system 100 according to certain forms of the technology; and

FIGS. 23A-C are perspective view illustrations of exemplary probe assemblies 200 according to other forms of the technology.

### 6. DETAILED DESCRIPTION OF EXEMPLARY FORMS OF THE TECHNOLOGY

### 6.1. Analysis System Overview

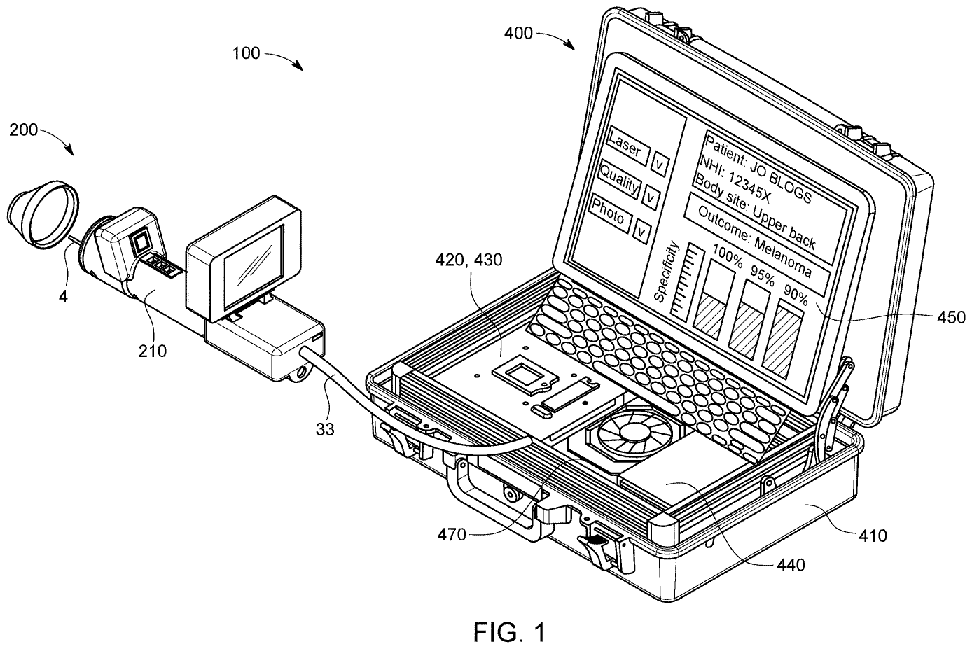

Forms of the technology relate to systems, devices and methods for analysing a subject, for example analysing a skin lesion on a patient. FIGS. 1, 2 and 21 illustrate exemplary analysis systems 100 for analysing a subject 500 according to certain forms of the technology. FIG. 1 is an illustration of one form of the technology illustrating the physical components of an exemplary analysis system. In the exemplary form, analysis system 100 comprises a probe assembly 200, a conduit 33 and a portable unit 400. Probe assembly 200 may comprise a probe 4 and a housing 210. The conduit 33 may comprise a plurality of light guides 310 (not shown in FIG. 1). The portable unit 400 may comprise a housing 410, a stimulus generator 420, a detector 430, a processor 440, an output device 450 and a power supply 470 (not shown). These components will be described in more detail below.

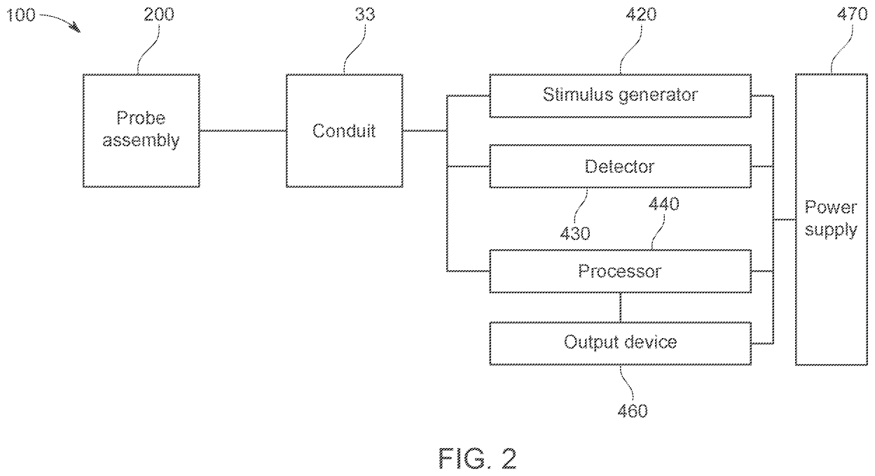

While FIG. 1 illustrates the physical form of an exemplary analysis system 100, FIG. 2 illustrates the functional aspects of an exemplary analysis system 100. In the exemplary form, analysis system 100 comprises a probe assembly 200, a conduit 33, a stimulus generator 420, a detector 430, a processor 440, an output device 450 and a power supply 470. Collectively, the probe assembly 200, the conduit 33, the stimulus generator 420 and the detector 430 may be considered to comprise a spectrometer. The spectrometer may also be considered to include the processor 440 and the output device 450, in some forms. Again, these components will be described in more detail below. FIG. 21 shows a schematic depiction of an alternative physical form of probe assembly 200 while a functional block 110 is illustrated to depict the stimulus generator 420, detector 430, processor 440, output device 450 and power supply 470, which are all components comprising the functional block 110. FIG. 21 also illustrates a power meter 620.

In some forms a functional aspect may be provided by one or more physical components. In some forms, one physical component may provide one or more functional aspects.

The analysis system 100 may be used to analyse a subject 500. Forms of the technology are not limited by the nature of the subject and, in some forms, any subject may be analysed by the system 100. The forms of the technology described herein may be particularly advantageously applied to analysing a region of skin and may be particularly useful for analysing the nature of lesions on human skin.

Therefore, in certain forms, the subject 500 is a skin region of a patient subject, which region may comprise a lesion on the skin of a patient. The analysis may generate, or result in, a diagnosis of a medical condition, for example skin cancer, or an indication that no such medical condition is present. While forms of the technology are particularly suitable for analysing a region of human skin, they may also be suitable for analysing regions of non-human animals. Some forms of the technology may additionally, or alternatively, be suitable for analysing tissue other than skin.

The different components of the analysis system 100 will now be described in more detail. It should be understood that, where different forms of each component of the analysis system 100 are described, each form may be interchangeably used with any of the forms of the other components described, even if that combination of components is not expressly described together, unless such a combination is clearly impractical.

Generally speaking, in certain forms of the technology, the analysis system 100 operates by generating light of a certain wavelength(s), illuminating the subject 500 with that light, collecting the light received from the subject 500 and analysing the spectrum of the collected light to determine one or more properties of the subject 500. For example, the analysis may result in the identification of the type of skin lesion that is the subject 500. The operation of exemplary analysis systems 100 will be described in more detail below.

It should be appreciated that, in this specification, the term “light” is intended to refer to electromagnetic radiation in general and may refer to any part of the electromagnetic spectrum. The term is not limited to optical light or any other region of the electromagnetic spectrum unless the context clearly indicates otherwise, or unless a narrower part of the spectrum is identified or a wavelength (or frequency) range is indicated.

### 6.2. Probe Assembly

In certain forms of the technology, the probe assembly 200 may be considered to be the physical device that is placed in close proximity to the subject 500 and used to expose the subject 500 to light and collect reflected light from the subject 500. Exemplary probe assemblies 200 are illustrated in FIGS. 1, 3 to 5, 13 to 21 and 23.

### 6.2.1. Housing

In certain forms, such as the illustrated examples, the probe assembly 200 comprises a housing 210. The housing 210 may generally form the outer surface of the probe assembly 200 or a substantial part thereof. The housing 210 may contain many of the components of the probe assembly 200 to protect them during use, may facilitate a user handling the probe assembly and may provide the probe assembly with an aesthetically pleasing appearance.

The housing 210 may be formed from a housing body 10 and a probe tip shield 7. The housing body 10 may provide, or contribute to, the functions of the housing 210 described above. The probe tip shield 7 may shield the tip 5 of the probe 4 (described below), for example substantially shielding the tip from ambient light and/or preventing the tip from unintended contact with nearby objects. Preventing ambient light from entering the probe 4 improves the accuracy of the spectral analysis undertaken by the analysis system 100. In certain forms, the probe tip shield 7 and housing body 10, collectively, may be configured to fully shield the tip 5 of the probe 4 from ambient light. In particular, these components may be configured to shield the tip 5 of the probe 4 from fluorescent light which, if detected by the detector 430 in the collected spectrum, may overwhelm the signal detected by the probe 4 and make spectral analysis difficult. This may be particularly the case when the probe assembly 200 is comprised as part of an analysis system 100 that performs Raman spectroscopy on subject 500, since ambient light may be particularly detrimental to the analysis in this case.

### 6.2.1.1. Housing Body

The housing body 10 may be a hollow body that is generally elongate in form and have a size and shape suitable for being held in a user's hand. For example, in some forms, such as shown in FIGS. 1, 3 to 5 and 13 to 23, the housing body 10 may be approximately cylindrical, or otherwise prism-shaped, although certain features of the housing body 10, or presence of other components, may cause the housing body 10 to deviate from such a geometric shape and this description should be understood to be a description of the housing body's approximate overall shape. The housing body 10 may be shaped to be gripped comfortably by a user, for example it may have an ergonomic design. In certain forms, the housing body 10 may be approximately 50 to 300 mm in length, and approximately 20 to 100 mm in width (or diameter). The housing body 10 may define one or more openings therein to allow components to pass through, or be positioned in, the walls of the housing body 10. The housing body 10 may be formed so as to be opaque in order to prevent ambient light entering the probe 4 during use. The housing body 10 may also be formed so as to be substantially rigid in order to provide structural rigidity to the probe assembly. For example, the housing body 10 may be formed from a hard and/or stiff material. In some forms, the housing body 10 may be formed from a plastic material, for example polycarbonate.

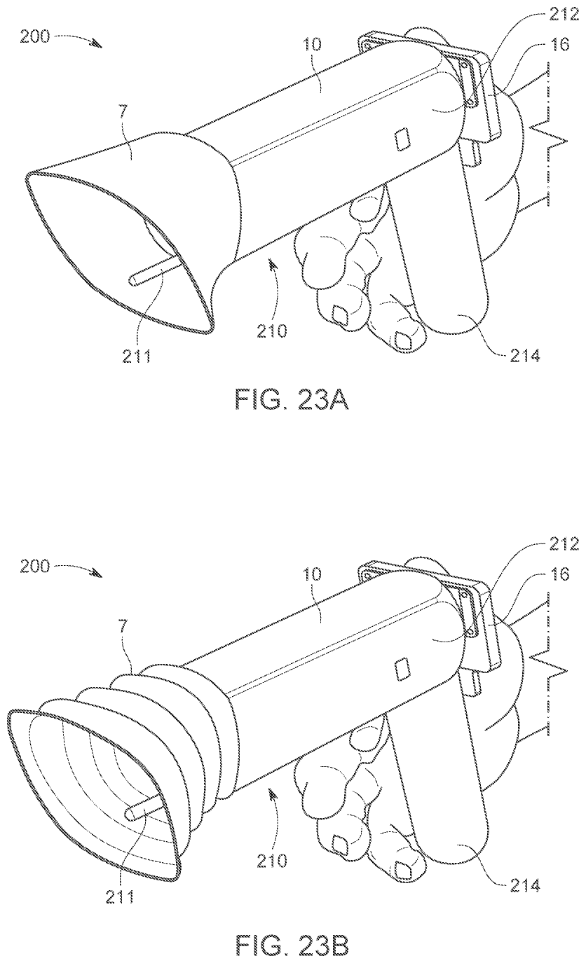

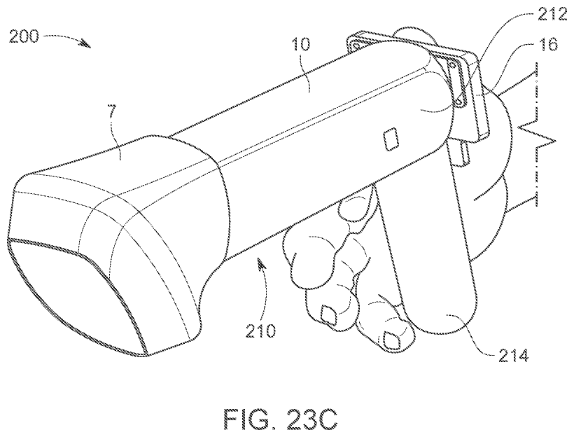

In some forms of the technology, for example as illustrated schematically in FIGS. 21 and 23, the housing body 10 may be formed with a general form factor resembling a gun. That is, the housing body 10 may comprise a generally cylindrical, or prism-shaped, body portion 212 and may also comprise a gripping portion 214 oriented at a non-zero angle to the body portion 212. The gripping portion 214 may be configured to be held by a user in order to handle and manoeuvre the probe assembly 200 during use. The probe assembly 200 may further comprise one or more control interfaces 215, which may comprise buttons, levers, switches, dials and the like, in a position on the housing body 10 convenient to be operated by a user when the probe assembly 200 is being held. For example, as shown in FIG. 21, a control interface 215 may be positioned on a part of the gripping portion 214 proximate to the body portion 214 on a side of the gripping portion 214 closest to the probe tip shield 7. For example, the control interface 215 may be positioned in the manner of a trigger in the form factor of a gun.

### 6.2.1.2. Probe Tip Shield

The probe tip shield 7 may be a hollow body provided to an end of the housing body 10, for example the probe tip shield 7 may connect adjacent to the end wall 31 of the housing body 10 through a friction fit, interlock, screw-fit connection, or any other suitable connection. For the purposes of describing the probe tip shield 7, it may be considered to have a proximal end and a distal end. In this context, “proximal” and “distal” are considered to refer to proximity of the respective ends of the probe tip shield 7 to the housing body 10, i.e. the proximal end of the probe tip shield 7 may be configured to connect to the housing body 10. The probe tip shield 7 may be configured to be removably connected to the housing body 10 so that the probe tip shield 7 can be disconnected from the housing body 10 and later re-connected, for example to allow cleaning or replacement of the probe tip shield 7.

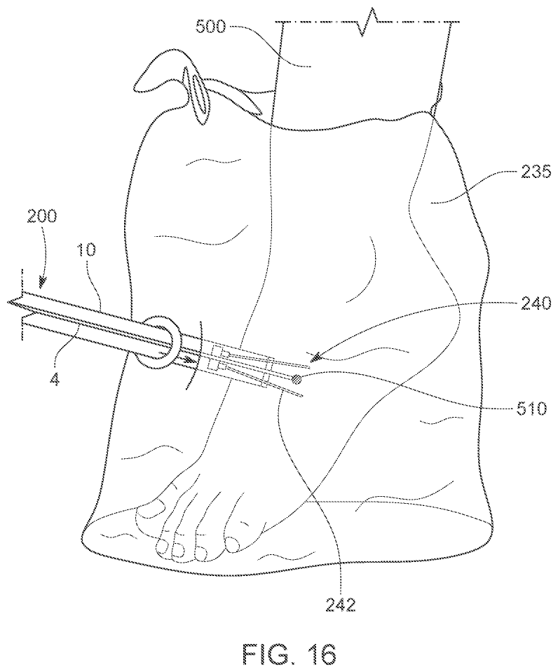

The proximal end of the probe tip shield 7 may have a width (or diameter) that is substantially similar to the width (or diameter) of the end of the housing body 10 to which the probe tip shield 7 connects. The length of the probe tip shield 7 may be less than, and in some forms significantly less than, the length of the housing body 10. For example, the length of the probe tip shield 7 may be approximately 10 to 70 mm in the case of the probe assembly 200 shown in FIGS. 1, 2 to 5, 13 to 15, 17 to 20 and 23. In the case of the probe assembly 200 shown in FIG. 16, the probe tip shield 7 may be larger, for example approximately 100 to 400 mm in length.

### 6.2.1.2.1. Taper

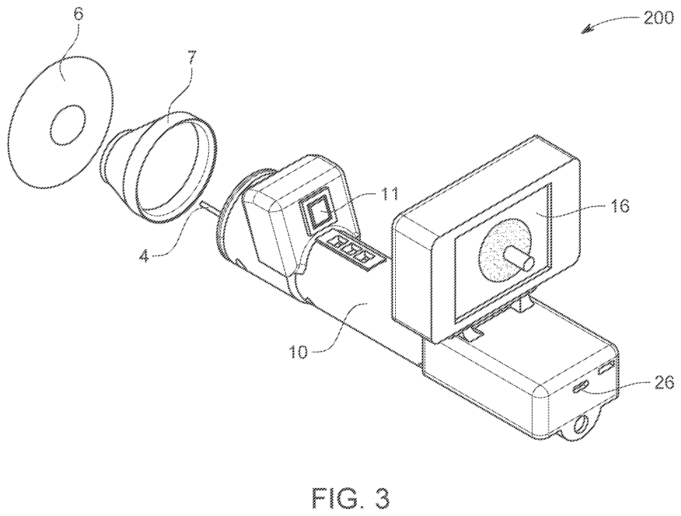

The probe tip shield 7 may be formed with a taper such that one of its ends is wider than its other end. In different forms of the technology, the taper may be in different directions, such as explained below. Examples of probe tip shields 7 according to certain forms of the technology are illustrated in FIGS. 3 to 7. In some forms, the probe tip shield 7 may comprise a body that tapers from the proximal end to the distal end, i.e. the proximal end may be wider than the distal end. In particular, the outer surface of the probe tip shield 7 may taper as described. In some forms, the probe tip shield 7 may be frustoconical, or substantially so. In other forms, the probe tip shield 7 may have another tapering shape, for example a frustum with substantially planar sides. The taper in the probe tip shield 7 provides a distal end that is thinner than the proximal end and this may enable a user to more accurately locate the probe to analyse the subject 500.

In certain forms, the proximal and distal ends of the probe tip shield 7 may each define an opening. In the case of a probe tip shield 7 that tapers as described above, the opening at the proximal end may have a larger area (e.g. be wider) than the opening at the distal end. In certain forms, the opening 220 at the distal end of the probe tip shield 7 may form a plane that is oriented generally perpendicularly to a longitudinal axis of the housing body 10. The opening at the proximal end of the probe tip shield 7 may also form a plane that is oriented generally perpendicularly to a longitudinal axis of the housing body 10, i.e. the planes formed by the openings at the proximal and distal ends of the probe tip shield 7 may be mutually parallel.

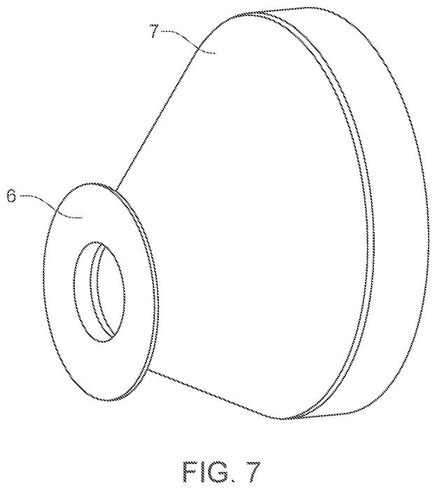

In some forms, for example as shown in FIGS. 6 and 7, the opening 220 at the distal end of the probe tip shield 7 may be radially offset from a longitudinal axis of the probe tip shield 7. In such forms, the probe tip shield 7 may be described as having the shape of a skewed frustum. This may be a beneficial arrangement if the probe 4 is not retained centrally within the probe assembly 200 in order for the tip 5 of the probe 4 to align with the opening 220 in the distal end of the probe tip shield 7.

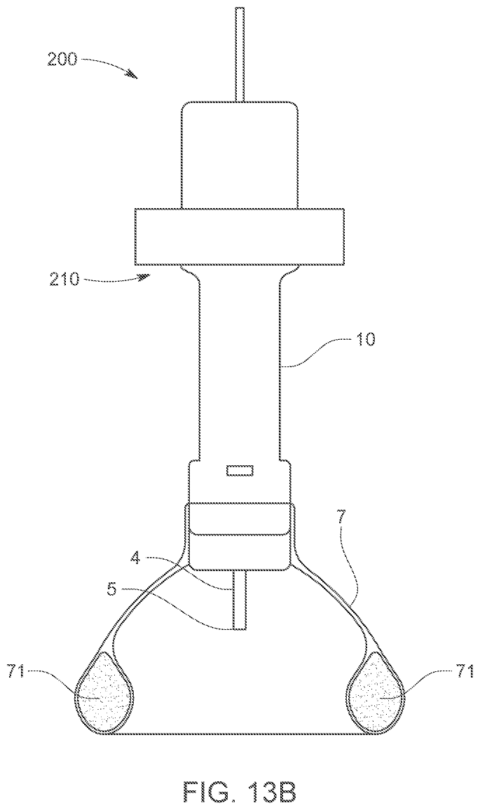

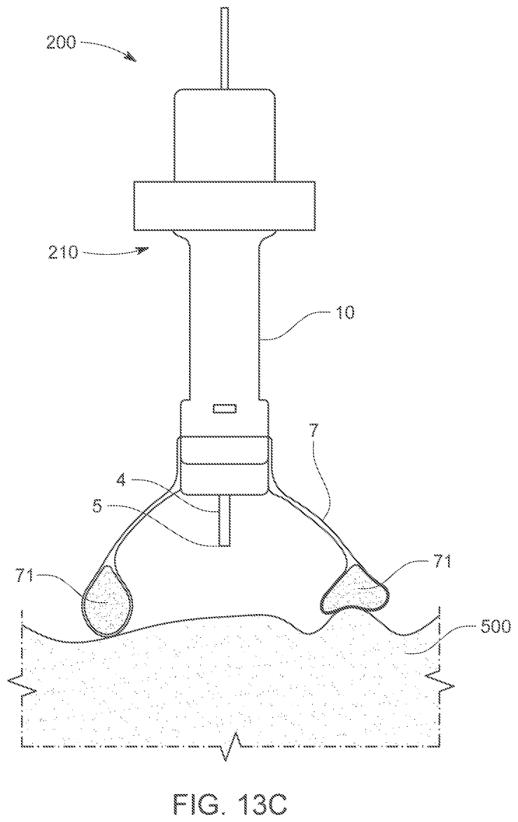

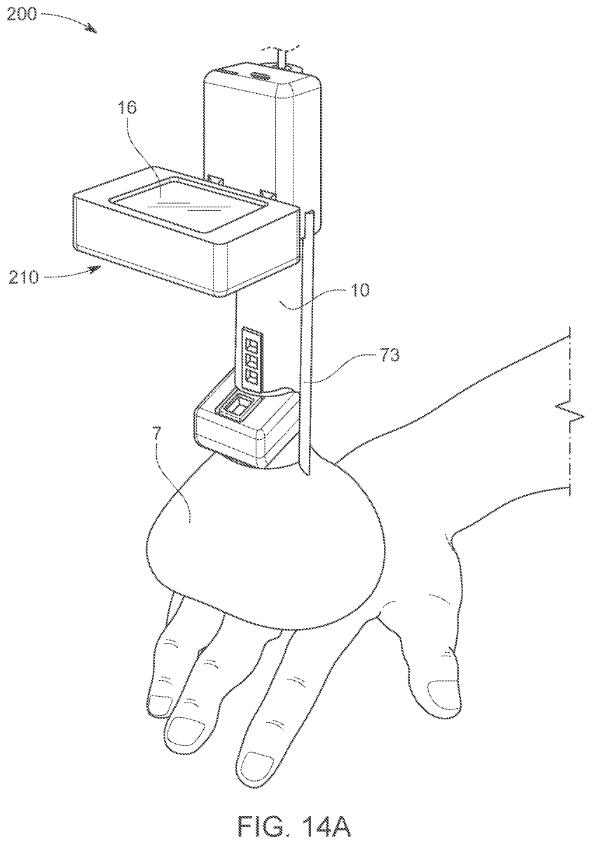

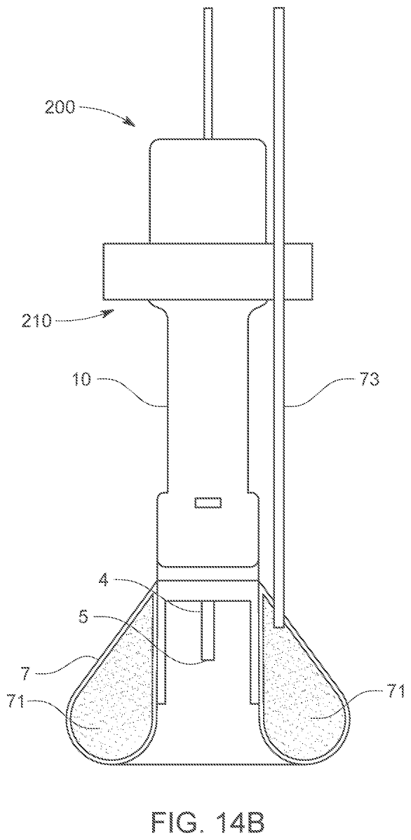

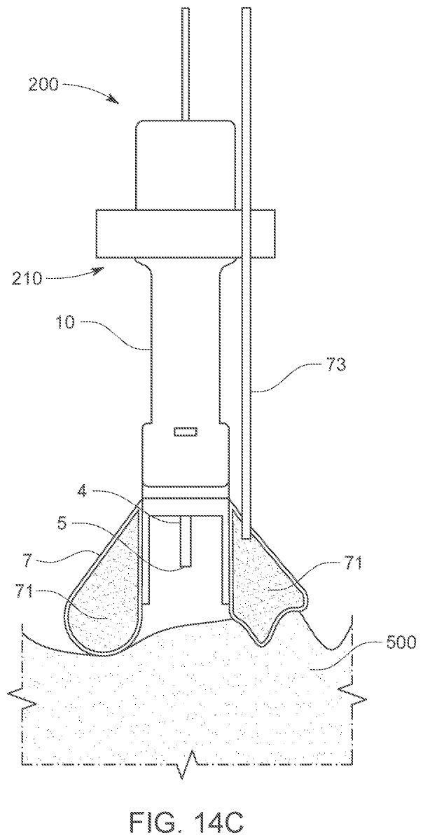

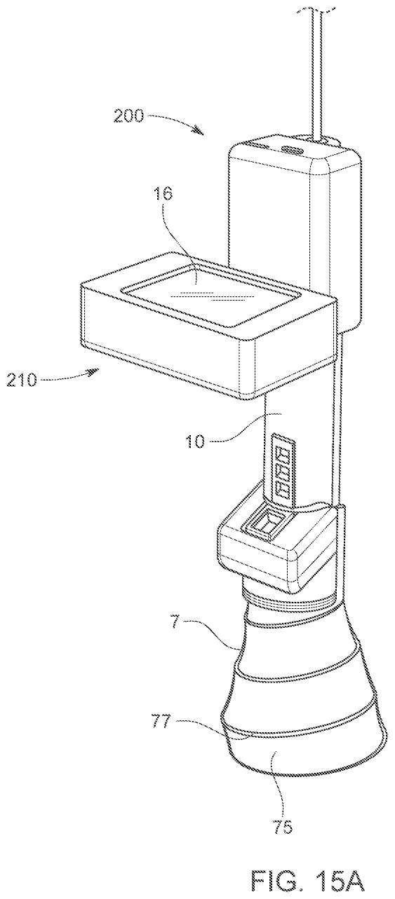

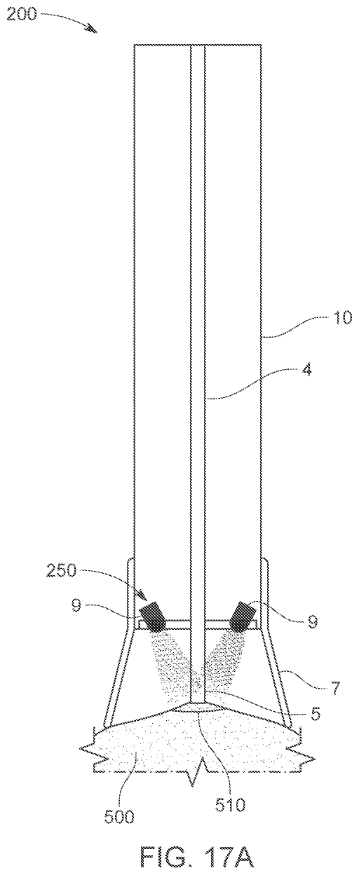

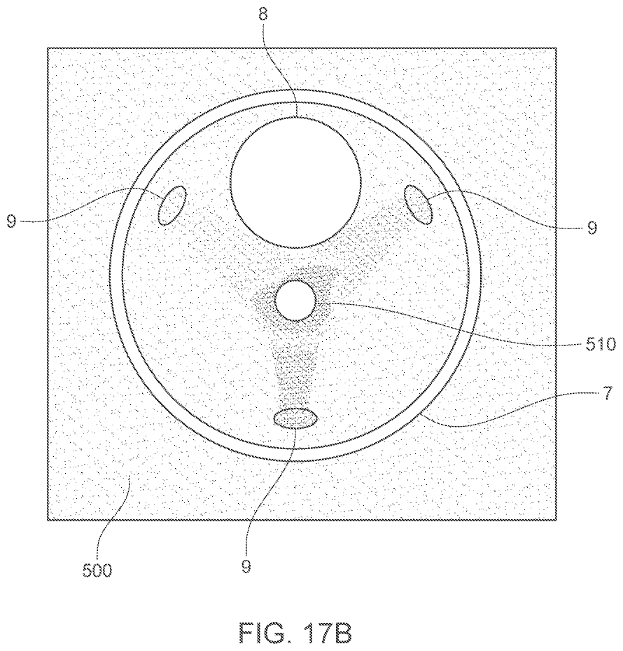

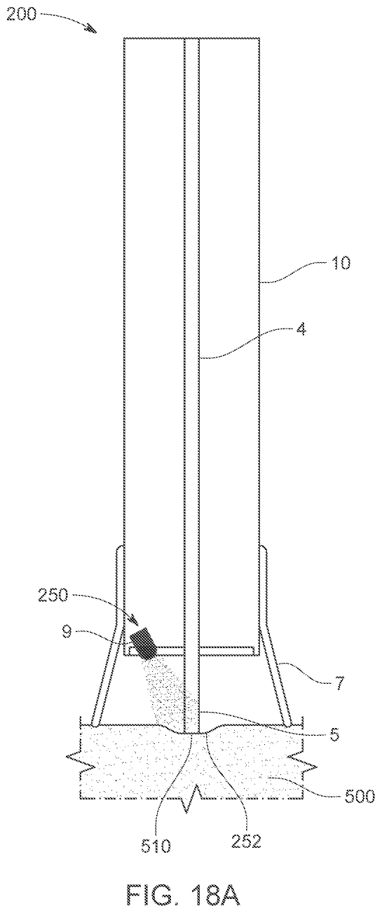

In other forms, for example as shown in FIGS. 13 to 15, 17 to 20 and 23, the probe tip shield 7 may comprise a body that tapers from the distal end to the proximal end, i.e. the distal end may be wider than the proximal end. The outer surface, the inner surface or both the outer and inner surfaces of the probe tip shield 7 may taper as described. In such forms, the opening at the distal end may have a larger area (e.g. be wider) than the opening at the proximal end, although depending on whether the taper is in the inner and/or outer surface of the probe tip shield 7, this may not necessarily be the case. For example, in the case of the probe tip shields 7 of the forms shown in FIGS. 17-20 and 23, the taper occurs in both the inner and outer surfaces so that the thickness of the probe tip shield 7 is substantially the same between its proximal and distal ends. Consequently, the opening at the distal end of the probe tip shield 7 is larger than the opening at the proximal end in these forms. In some forms, for example in the case of the probe assembly 200 shown in FIG. 23A, the angle of the wall of the probe tip shield 7 with a longitudinal axis of the housing body 10 may increase from the proximal end to the distal end of the probe tip shield 7. For example, as shown, the probe tip shield 7 may have a shape that flares outwardly towards its distal end. In some forms, the degree of flaring may be the same around the circumference of the probe tip shield 7, while in other forms, the degree of flaring may differ around the circumference. In the case of the probe tip shield 7 shown in FIGS. 14A-C, the taper occurs in the outer surface but the inner surface does not taper so that the thickness of the probe tip shield 7 substantially increases towards the distal end. Consequently, the opening at the distal and proximal ends of the probe tip shield 7 are substantially similar in size in this form. In the case of the probe tip shield 7 shown in FIGS. 13A-C, the taper occurs in the outer surface and in a proximal region of the inner surface. In the distal region, the thickness of the probe tip shield 7 is substantially larger than at the proximal end and the distal region of the inner surface tapers a little to narrow the opening, but overall the opening at the distal end of the probe tip shield 7 is larger than the opening at the proximal end in this form. Similarly, in the case of the probe tip shield 7 shown in FIG. 23C, there is a first region of tapering at the proximal end in which the width of the shield increases away from the housing body, and a second region of tapering at the distal end in which the width of the shield decreases away from the housing body. The amount of tapering in the first region is greater than the amount of tapering in the second region so that the opening at the distal end of the probe tip shield 7 is larger than the opening at the proximal end in this form. In certain forms, the width of the probe tip shield 7 at its distal end, which may be its diameter in the case of probe tip shields with substantially circular distal ends, may be in the range of approximately 15 to 150 mm, for example at least 50 mm. This width and/or diameter may correspond to the width/diameter of a region of the subject's body substantially shielded by the probe tip shield 7 during use of the probe assembly 100. In some cases, the shape of the probe tip shield 7 may be non-circular. In these forms, the region of the subject's body substantially shielded by the probe tip shield 7 during use may vary around the perimeter of the region. In such cases, the minimum width may be in the range of approximately 15 to 150 mm, for example at least 30 mm, for example at least 50 mm.

A taper in this direction may be useful so that the distal end of the probe tip shield 7 covers a relatively large region of the subject's skin compared to the width of the housing body. With some forms of probe 4, ambient light may enter the probe 4 because the subject 500 is not entirely opaque and some ambient light is able to pass through the subject 500, e.g. through sub-surface scattering, and enter the tip 5 of the probe 4. Such light may be referred to as interactance light. Since ambient light may have an adverse effect on the accuracy of the analysis performed by the analysis system 100, including in the case of Raman spectroscopy, the probe assembly 200 may be configured to reduce the amount of interactance radiation entering the probe 4. A probe tip shield 7 that covers a relatively large area of the subject's skin around the region of the skin being analysed may achieve this. For example, a minimum width of approximately 50 mm (5cm) has been found to sufficiently shield from ambient light for the purposes of Raman spectroscopy performed on human skin. The minimum suitable width may vary depending on various factors, including the degree of sub-surface scattering in the surface being analysed and the intensity of the illuminating light. The minimum suitable width for specific applications of the probe assembly 200 may be able to be determined by experimentation as to the width of distal end of the shield that reduces interactance light to a sufficient level for the type of spectral analysis being performed. In certain forms, the probe tip shield 7 is configured such that the region of the subject's body surrounding the skin region 500 has a minimum width sufficient to substantially eliminate sub-surface scattered ambient light being received by the probe 4.

### 6.2.1.2.2. Opacity

The probe tip shield 7 may be formed so as to be substantially opaque in order to prevent ambient light entering the probe 4 during use. When the probe assembly 200 abuts against the subject 500 so that the distal end of the probe tip shield 7 is in contact with the subject 500 all around the opening 220, the opaque probe tip shield 7 and housing body 10 together substantially or fully prevent ambient light from entering the tip 5 of the probe 4 during use.

In particular, the probe tip shield 7 is substantially opaque to light of wavelengths of sensitivity to the type of spectroscopy performed by analysis system 100. For example, in the case of Raman spectroscopy being performed, the probe tip shield 7 may be at least substantially opaque to light of wavelengths of approximately 200-2000 nm in order to shield ambient light from entering the probe 4 that may impact on the spectral analysis. In some forms, the probe tip shield 7 may be substantially opaque to light at substantially all wavelengths.

The degree of opacity of the probe tip shield 7 may be sufficient to reduce the amount of ambient light transmitted through the shield to a low enough level that the noise in the detected spectrum from the ambient light is sufficiently low so as not to affect the results of the analysis. The appropriate degree of opacity may therefore vary dependent on the particular instance of use of the probe tip shield. In some forms, the optical density of the probe tip shield 7 may be approximately 2 or higher, and in some forms may be approximately 7 or higher.

The desired opacity of the probe tip shield 7 may be achieved through selection of a suitable material or materials used to form the probe tip shield 7 and/or the physical structure of the shield. In some forms, the probe tip shield 7 may be coated with a substance enhancing the opacity of the shield, e.g. light-blocking paint.

### 6.2.1.2.3. Conformability

In certain forms, the probe assembly 200 is configured such that a shape of the probe tip shield 7, for example a shape of the distal end of the probe tip shield 7, is able to be adapted to conform to a region of the subject's body surround the skin region under analysis. Conforming the probe tip shield 7 to the subject's skin in this way may improve the shielding of ambient light by avoiding light entering into the volume under the probe tip shield 7 through gaps between the probe tip shield 7 and the subject. The probe assembly 200 may be configured in a number of different ways to achieve this conformability, as will now be described.

In certain forms, for example as is the case with the probe tip shields 7 shown in FIGS. 13-16 and 23, the probe tip shield 7 may be configured so as to be flexible in shape so that its distal end can conform to a region of the subject's skin. Flexibility in the probe tip shield 7 may be achieved through the structure of the probe tip shield 7 and/or the material from which it is made.

For example, in some forms, such as shown in FIGS. 13-16 and 23, the probe tip shield 7, or at least a distal end of the probe tip shield 7, may be formed with walls having a thickness that is significantly less than the size of the walls in length and/or width. This may promote flexibility in the shape of the probe tip shield 7.

Additionally, or alternatively, the probe tip shield 7, or at least a distal end of the probe tip shield 7, may be formed from a material that promotes flexibility, e.g. a material that is relatively soft and/or has a relatively high elastic modulus. Examples of suitable materials may include elastomers, for example silicone rubber, thermoplastic elastomers, rubbers, etc. It may be particularly desirable for the material to be biocompatible in order to avoid causing harm to the subject's living tissue that it comes into contact with during use.

In some forms, for example in the forms of FIGS. 13 to 15 and 23, the probe tip shield 7 may comprise one or more flaps and/or folds to provide flexibility to the shield. In some forms, the distal end of the probe tip shield 7 may comprise a flap that folds over, either radially inwardly or radially outwardly to create a cushion that can flex and conform to a subject's skin. In some forms, such as in the forms of FIGS. 13 and 14, the flaps may fold over and connect with another portion of the probe tip shield 7 so as to form one or more cavities 71 in the distal end of the shield. The walls of the cavities 71 may be flexible so as to facilitate the distal end conforming to the region of the subject's body surrounding the skin region, such as is shown in FIGS. 13C and 14C. The cavities may be filled with a fluid, for example air or gel, or a granular material, for example small pellets or granules, to permit the probe tip shield 7 to change shape when a force is imparted on the walls of the cavities, e.g. the reaction force when the probe tip shield 7 comes into contact with the surface of the subject.

In some forms, for example as shown in FIGS. 14A-C, the rigidity of the cavity 71, and consequently the rigidity of the probe tip shield 7, may be adjustable by alteration of the pressure of the substance in the cavity 71. The probe assembly 100 may further comprise a tube 73 with one end inserted into the cavity 71 through the walls of the probe tip shield 7 and the other end connected to a pump (not shown) configured to inject fluid into, or extract fluid from, the cavity 71, for example a vacuum pump. There may be a seal around the perimeter of the tube 73 where it is inserted into the cavity 71 to avoid leaks. The tube 73 may be oriented substantially parallel with housing body 10 to enable the probe assembly 100 to be easy to handle by a user and, in some forms, the tube 73 may be housed within housing body 10. The pump may be comprised as part of the probe assembly 100 or the pump may be comprised as part of portable unit 400, in which case the tube 73 may extend along the conduit 33. In use, before and/or after the probe tip shield 7 has been located in the desired position against the subject's skin, the pump may be activated to achieve the desired level of rigidity of the probe tip shield 7. In one form of the example shown in FIGS. 14A-C, the probe tip shield 7 may function in the manner of a jamming gripper. In this form, the cavity 71 formed interior to the walls of the probe tip shield 7 may be filled with a granular material. Use of a vacuum pump on the cavity 71 after the probe tip shield 7 has been located in the desired position against the subject's skin may cause the granules in cavity 71 to be sucked together under the negative pressure created by the pump, resulting in the probe tip shield 7 solidifying and acting to grip against the subject's skin.

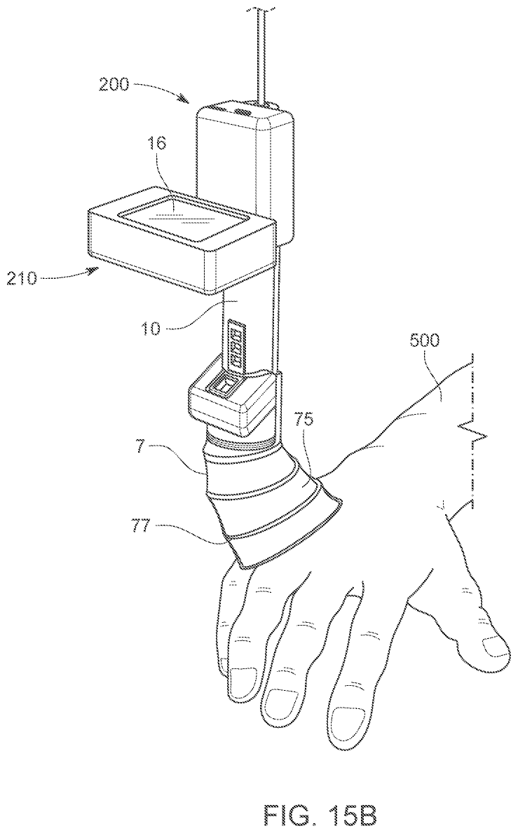

In forms such as shown in FIGS. 15A and 15B, 23B and 23C, the probe tip shield 7 may comprise one or more folds enabling the probe tip shield 7 to deform in shape to facilitate its distal end conforming to the subject's body. In the example of FIGS. 15A and 15B, the probe tip shield 7 may comprise a flexible canopy 75 and one or more frame members 77 providing structure to the canopy 75. The canopy 75 may be formed of a sheet-like flexible material. The frame members 77 may be formed so as to be relatively rigid, e.g. they may be formed from a relatively hard or inelastic material. In some forms, for example as shown in FIG. 23B, the probe tip shield 7 may comprise a plurality of folds in the manner of a concertina, for example the canopy 75 may fold in between the frame members 77. The folds may be oriented circumferentially around the shield 7, and may be oriented perpendicularly to the longitudinal axis of the housing body 10. The folds may be substantially mutually parallel. Additionally, or alternatively, the folds may be substantially equally spaced longitudinally along the shield 7 between the proximal and distal ends. In other forms, the folds may be oriented at a different angle to the longitudinal axis and each of their orientations may differ. In other forms, the spacing between each or some of the folds may differ. The presence of folds may enable the probe tip shield 7 to be flexible in shape and optionally to fold up for storage. As shown in FIGS. 15A and 15B, the probe tip shield 7 may comprise a single frame member 77 formed in a helical shape, for example a helical spiral where the radius of the helix increases with distance from the proximal end of the probe tip shield 7 to provide a taper to the shield as explained earlier.

In some forms, for example as shown in FIG. 16, the probe tip shield 7 may be formed from one or more sheets of material assembled so as to form an enclosure 235, for example a bag or box. The enclosure 235 comprises an opening at the proximal end connected to the housing body 10 and an opening at the distal end. The enclosure 235 may comprise a closure mechanism to close the opening at the distal end around part of the subject's body, for example a drawstring, zip, press seal or the like, in order to substantially prevent light entering the enclosure. The enclosure 235 may be sufficiently large that a part of the subject's body, for example a foot, head, arm, leg or head, may be contained within the enclosure 235. The enclosure 235 may be formed from a material that shields ambient light from entering into the enclosure, at least at wavelengths of sensitivity for Raman spectroscopy and in some forms all wavelengths.

In other forms, the probe tip shield 7 may comprise a plurality of pins, arranged in parallel to each other and oriented substantially parallel to a longitudinal axis of the probe assembly 200. The pins may be mounted to a base member such that each pin is able to slide longitudinally. The ends of the pins at the distal end of the probe tip shield 7 may form a surface that is able to conform to different shapes, and consequently conform to different parts of the subject's body in use. In some forms, the position of the pins may be able to be locked into place once the desired configuration has been achieved.

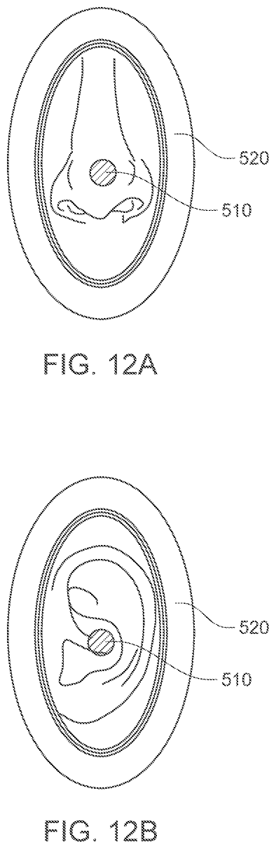

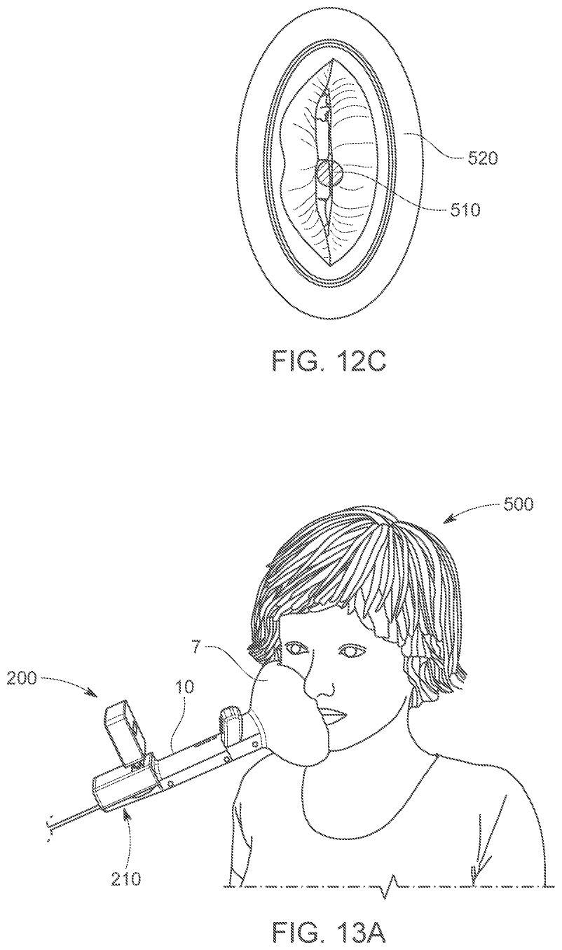

In some forms, the probe tip shield 7 may be configured to fit around a specific body part. For example, the probe tip shield 7 may be formed with a specific shape and size to fit around a body part that is commonly a site where a skin region needing to be analysed is located, or proximate such a site. That is, the probe tip shield 7 may be formed so that its distal end surrounds a particular body part. FIGS. 12A-C illustrate in plan view regions of a subject's body that may be spectrally analysed using a probe assembly 200 according to certain forms of the technology. Skin region 510 is the region being spectrally analysed by the probe 4. In the case of FIGS. 12A-C, skin region 510 is located on a protruding part of the subject's body, for example their nose (FIG. 12A), ear (FIG. 12B) and mouth (FIG. 12C). The contact region 520 shown in these figures is the region of the subject's body surrounding the skin region 510 against which the distal end of the probe tip shield 7 abuts when the probe assembly 200 is in use. The probe tip shield 7 may be configured so that it has a size and shape suitable to create a contact region 520 that surrounds the relevant body feature (e.g. nose, ear, mouth) and may in particular contact relatively flat regions of the body around these features. This may promote an effective light seal between the probe tip shield 7 and the subject's body. In some forms, a single probe tip shield 7 may be formed in a shape and/or size that makes it suitable to surround a number of different body features, for example each of the types shown in FIGS. 12A-C. In other forms, different probe tip shields may be suitable for use with different body features. In some forms, the probe tip shield 7 may be particularly configured to be used to surround a particular body feature by being formed with a shape such that the inner walls of the probe tip shield 7 complement the shape of the body feature. For example, the inner walls of the probe tip shield 7 may be formed with a shape that is the negative of the three-dimensional shape of the relevant body part, or approximately so to a subdued extent.

In some forms, the probe assembly 200 may comprise a plurality of interchangeable probe tip shields 7. Each probe tip shield 7 may be different in one or more properties, for example each may have a different shape, size, opacity and/or flexibility. In some cases, the distal end of each probe tip shield 7 may differ in such a way. Each probe tip shield 7 may comprise a proximal end configured to be interchangeably attached to the housing body 10 of the probe assembly 200. Such interchangeability may be useful to enable selection of a probe tip shield 7 suitable for the particular skin region 510 to be analysed, for replacement of the probe tip shield 7 and/or for cleaning of the probe tip shield 7. The proximal end of each probe tip shield 7 may be removably attached to the housing body 10 using any suitable mechanism, such as described above.

In other forms, the probe tip shield 7 may be formed temporarily for a limited number of uses of the probe assembly 200. For example, in some examples, the probe assembly 200 may be put into the desired position relative to the subject and then a substance may be introduced around the subject-proximal end of the probe assembly 200 to form a light-proof seal against the subject 500 and consequently form the probe tip shield 7. For example, a substance such as a fluid, gel, paste, putty or granular mass may be added around the end of the probe assembly. The substance may set or cure after a time, keeping it in place for the use of the probe assembly 200. These substances, being fluid or malleable in their original states, may be able to form a probe tip shield 7 that caters for complex shapes on the surface of the subject's body.

### 6.2.1.3. Skirt

In some applications of the probe assembly 200, some ambient light may still be able to enter the probe 4 even if the distal end of the probe tip shield 7 is positioned to abut the subject 500. In some cases, this may be because the user has not quite positioned the distal end of the probe tip shield 7 exactly flush with the subject 500. In these cases, reduction of ambient light entering the housing 210 can be improved by better positioning of the probe assembly 200 relative to the subject 500. In other cases, ambient light may enter the probe 4 because the subject 500 is not entirely opaque and some ambient light is able to pass through the subject 500, e.g. through sub-surface scattering, and enter the tip 5 of the probe 4. Such light may be referred to as interactance light. Such ambient light may have an adverse effect on the accuracy of the analysis performed by the analysis system 100.

It has already been described how, in some forms, this ambient light may be reduced by a relatively large width of the distal end of the probe tip shield 7. In some forms, the probe assembly 200 may comprise one or more additional components configured to reduce the amount of interactance radiation entering the probe 4. In the exemplary forms of the technology illustrated in FIGS. 3 to 5, the probe assembly 200 comprises a skirt 6 with this purpose. The skirt 6 may be a body of material that extends radially outwardly from the distal end of the probe tip shield 7 so that, when the distal end of the probe tip shield 7 is placed against the subject 500, the skirt 6 covers a surface region substantially surrounding the subject 500, or the region of the subject 500 being analysed, and consequently reduces the amount of ambient light that is able to enter the surface region surrounding the subject 500. In turn, this reduces the amount of light that can scatter through the subject 500 and enter the probe 4. In certain forms, the skirt 6 may extend substantially radially perpendicularly outward from the probe tip shield 7, i.e. in a perpendicular direction from the longitudinal axis of the probe assembly 200, e.g. in the manner of a flange. In some forms, the skirt 6 may be angled slightly forward (i.e. away from the housing body 10) of the perpendicular direction to assist with covering the surface of the subject 500, especially if that surface is uneven.

In the illustrated forms, the skirt 6 comprises an annular body with the inner edge of the annular body being provided to the outer surface of the distal end of the probe tip shield 7. Consequently, the shape of the hole in the skirt 6 may be configured to match the shape (and size) of the outer surface of the distal end of the probe tip shield 7. In some forms, the skirt 6 may be provided to the probe tip shield 7 through a friction fit, interlock, screw-fit connection, or any other suitable connection. In other forms, the skirt 6 and probe tip shield 7 may be integrally connected, for example formed as a single body. The skirt 6 may be a substantially planar body with a depth significantly smaller than its width. While the skirt 6 is illustrated as a circular annulus in the figures, in other forms, the skirt 6 may have a different plan-view shape, for example oval, square, rectangular, each with a hole formed therein, or other suitable configuration.

In some forms, the distance that the skirt 6 extends outwardly away from the distal end of the probe tip shield 7 may be in the range of approximately 5 to 50 mm or larger in some forms. If the width of the skirt 6 is considered its dimension from one side to another, e.g. its diameter in the case of a circularly annular skirt 6, the width may be in the range of approximately 15 to 150 mm, i.e. a radially outer edge of the skirt 6 has this width/diameter. A larger sized skirt 6 may be used where the subject 500, or the region surrounding the subject 500, has a greater transparency. As explained earlier, a minimum width of approximately 50 mm has been found to provide a sufficiently effective shield for Raman spectroscopy conducted on the skin of a human. In some forms, a radially outer edge of the skirt 6 has a minimum width sufficient to substantially eliminate sub-surface scattered ambient light being received by the probe 4.

The skirt 6 may be formed so as to be substantially opaque in order to prevent ambient light being incident on the region surrounding subject 500. The opacity may be at least at the wavelengths of sensitivity to the type of spectroscopy being performed, e.g. Raman. In some forms, the skirt 6 may be formed so as to be flexible or semi-rigid so as to be able to flex when pushed up against subject 500, for example the skirt 6 may be formed from a relatively soft and/or elastic material, or the skirt 6 may be formed to be sufficiently thin to allow flex therein. In other forms, the skirt 6 may be formed to be rigid, for example the skirt 6 may be formed from a hard and/or stiff material, or the skirt 6 may be sufficiently thick to provide rigidity. In some forms, the skirt 6 may be formed from a plastic or elastomer material, for example polycarbonate or silicone.

In some forms, the skirt 6 may be detachably connected to the probe tip shield 7 so that the skirt 6 may be removed from the probe tip shield 7. In such forms, skirts of different sizes may be able to be selectively mounted to the probe tip shield 7. An appropriately sized skirt may be selected for the intended use. Interchangeable skirts formed of different materials or shapes, for example skirts having different degrees of transparency or different rigidities, may also be provided.

In some forms, the probe tip shield 7, the skirt 6 and/or an assembly comprising the probe tip shield 7 and the skirt 6 (in which the probe tip shield 7 and skirt 6 may be integrally formed as a single component) is interchangeably mountable to the end of the housing body 10. This component may be interchanged for each patient so that a clean component is used to contact the patient each time. Any suitable interchangeable mounting may be used, for example friction fit, interlock, screw-fit connection, or any other suitable connection.

### 6.2.1.4. Spacer

In certain forms of the technology, the probe assembly 200 may comprise a spacer 211. Spacer 211 may be configured to facilitate positioning the probe assembly a desired distance from the subject 500, and particularly skin region 500, in use. While the probe assembly 200 may be configured with the ability to retract and extend the probe 4 in order to carefully position its tip relative to the subject (exemplary retraction mechanisms are described later), it may additionally or alternatively be advantageous to regulate the positioning of the probe assembly 200, and in particular the housing body 10, relative to the subject 500. In some forms, the spacer 211 may act as a coarse positioning guide for the user to approximately position the probe assembly 200 relative to the subject, and the retraction mechanism may facilitate more careful positioning and alignment of the probe with the skin region.

In some forms, for example as shown in FIGS. 23A and 23B, the spacer 211 may project out of a patient-proximal end of the housing body 10. For example, the spacer 211 may project out of the end wall 31 of the housing body. The spacer 211 may project outwardly from the housing body 10 from a point offset from the opening 28 through which the probe 4 extends through the wall of the housing body 10. The proximal end of the spacer 211 may be close to, e.g. immediately adjacent, the opening 28. In some forms, the spacer 211 may be connected to the housing body 10 through a suitable connection mechanism, while in other forms the spacer 211 may be integrally formed with the housing body 10. As shown in FIGS. 23A and 23B, the spacer 211 may project out of the end wall 31 of the housing body 10 in a direction substantially parallel to the longitudinal axis of the housing body 10. In other forms, the spacer 211 may project at a non-zero angle to this axis. The spacer 211 may take the form of an elongate member, such as a rod. The spacer 211 may be configured to be rigid or semi-rigid so that, when the probe assembly 200 is brought closer to the subject in use, the spacer 211 abuts against the subject's skin in a way that can be felt by the user holding the probe assembly 200 and indicates that the probe assembly 200 should not be moved any closer to the subject. The rigidity of the spacer 211 may be achieved through selection of the shape of the spacer 211, e.g. it may be rod-like in shape, or the material used to form it, e.g. it may be formed from one or more hard or rigid materials, or both.

In some forms, such as shown in FIGS. 23A and 23B, the spacer 211 may be positioned inside probe tip shield 7. In other forms, the spacer may be positioned outside the probe tip shield 7 or may be comprised as part of the probe tip shield 7. For example, in some forms, the spacer 211 may be formed as part of, for example embedded within, probe tip shield 7.

While only a single spacer 211 has been described above and is shown in FIGS. 23A and 23B, in other forms, the probe assembly 200 may comprise a plurality of spacers 211 of any one or more of the forms described above. The plurality of spacers 211 may be arranged around the probe 4, for example on opposite sides of the probe.

### 6.2.2. Probe

In certain forms of the technology, the probe assembly 200 comprises a probe 4. The probe 4 may be configured to illuminate the subject 500 with electromagnetic radiation (i.e. light) and to collect light from the subject 500 in use. Light may illuminate the subject 500 from, and collect light from the subject 500 at, a tip 5 at one end of the probe 4. The tip 5 may be referred to as the subject-proximal end of the probe 4 since it is placed proximal to the subject 500 in use. The other end of the probe 4 may be referred to as the subject-distal end of the probe 4 as it is located further from the subject 500 during use.

### 6.2.2.1. Light Guides

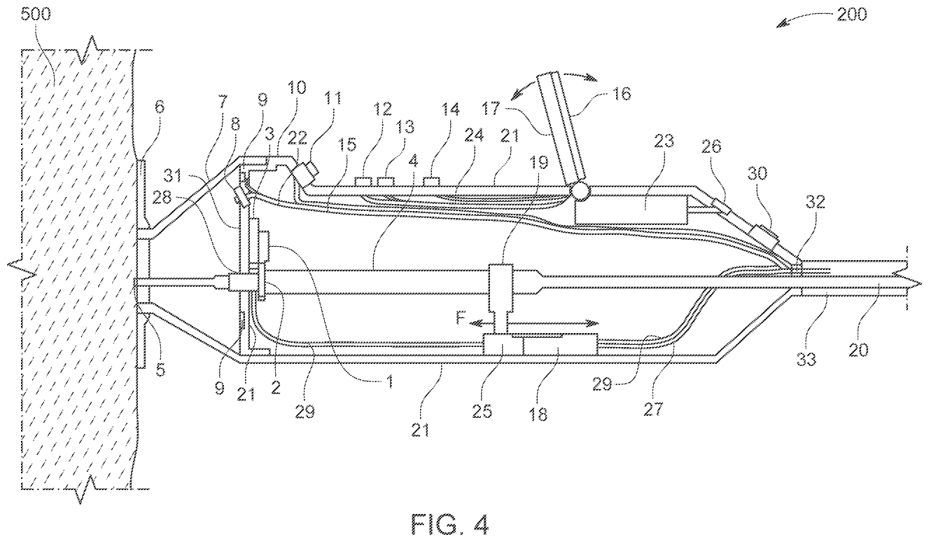

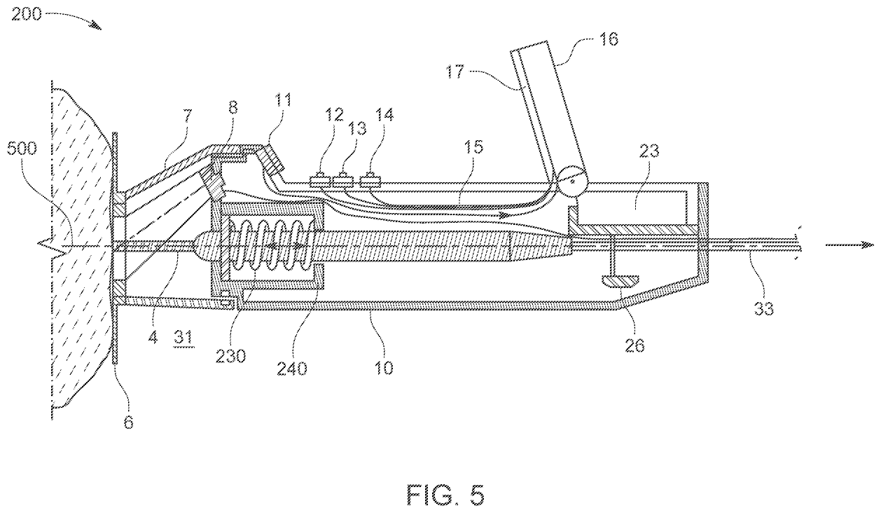

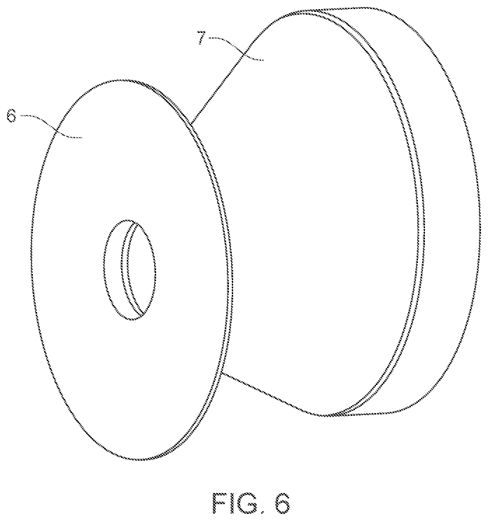

The probe 4 may be a structure configured to convey light from one location to another. In certain forms, the probe 4 may comprise a plurality of light guides 310 which function to convey light. The light guide may be fibre optic cables, for example. The plurality of light guides 310 may comprise multiple types of light guides. A first type, which will be referred to as illuminating light guides 312, are configured to convey light from the subject-distal end of the probe (on the right hand side of FIGS. 4 and 5) to the subject-proximal end of the probe (on the left hand side of FIGS. 4 and 5) where light exits the probe 6 and may illuminate the subject 500. A second type, which will be referred to as collecting light guides 314, are configured to collect light from the subject 500 and convey the collected light from the subject-proximal end of the probe to the subject-distal end of the probe where light exits the probe 6, for example passing into a conduit 33. The probe 4 may comprise one or more of each type of light guide, for example between one and 50 light guides.

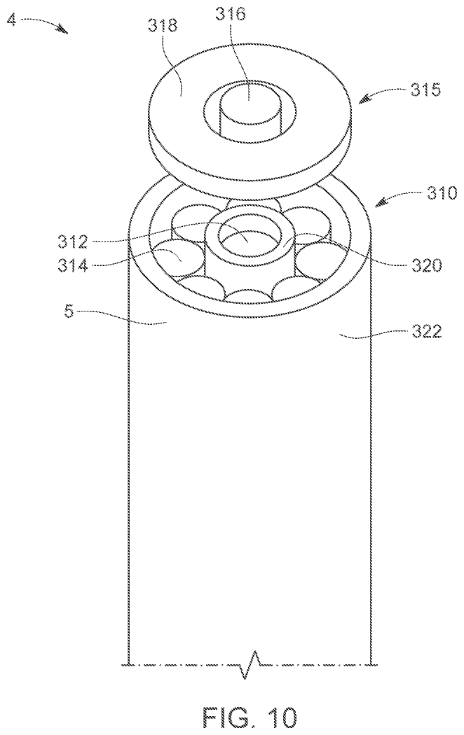

FIG. 10 illustrates the tip 5 of an exemplary probe 4 according to one form of the technology. In this form, a single illuminating light guide 312 is positioned axially centrally within the probe 4. The illuminating light guide 312 may be wrapped in an opaque inner sheath 320. Positioned circumferentially around the illuminating light guide 312 are a plurality of collecting light guides 314. In the example of FIG. 10, there are eight collecting light guides 314 but this number may differ in other forms (and other forms may also have different numbers of illuminating light guides 316). The collecting light guides 314 may be wrapped in an opaque outer sheath 322, which may also be, or form part of, the outer casing of the probe 4.

### 6.2.2.2. Filters

The subject-proximal end of the probe 4 that contacts the subject 500 in use may further comprise one or more filters 315 configured to remove unwanted wavelengths of light. The one or more filters 315 may comprise one or more illumination filters 316 and/or one or more collecting filters 318. For example, in some applications of the technology a light source from the excitation fibre 312 having a wavelength of approximately 830 nm may be used, and it may be advantageous to use a filter 316 with a specific bandpass filter to allow only light with a wavelength of approximately 830nm to excite the subject. In some examples, it may be advantageous to use a filter 318 to ensure that only collected light that has originated from the subject that has been scattered by the light source is detected by the detector 430. For example, in some applications a light source from the excitation fibre 312 having a wavelength of approximately 830 nm may be used, and it may be beneficial to exclude light close to this wavelength by using a bandpass filter that attenuates light below a predetermined wavelength, for example approximately 840 nm, so that only collected light longer than this wavelength that has originated from the subject that has been scattered by the light source is detected by the detector 430. Additionally, or alternatively, a filter may also attenuate light above another predetermined wavelength. Filters 315 may be positioned adjacent the light guides 310 so that, in use, the filters are positioned between the probe 4 and the subject 500. In some forms, filters 315 are positioned directly adjacent the tips of the light guides 310, i.e. the filters abut the ends of the light guides. In the exemplary form illustrated in FIG. 10, the filters comprise an illuminating filter 316 and a collecting filter 318. The illuminating filter 316 may be positioned between the illuminating light guide 312 and the subject 500 in use, e.g. directly adjacent the tip of the illuminating light guide 312. The illuminating filter 316 may be a cylindrical member. The collecting filter 318 may be positioned between the collecting light guides 314 and the subject 500 in use, e.g. directly adjacent the tips of the collecting light guides 314. The collecting filter 318 may be an annular-shaped member.

### 6.2.2.3. Transmission Window

In some examples, the subject-proximal end may additionally, or alternatively, include one or more transmission windows. The transmission window may be located at the subject-proximal end of the probe 4 and provide an interface between the stimulus (light) generated by the stimulus generator 420 and the subject 500 in use. In addition, the interface may allow for incident light to be detected by the detector 430. The transmission window may be constructed from any suitable light transmission medium. It may be desirable for the transmission window to be formed of a material with a low nonlinear refractive index, and a transmission range wide enough to cover the range of frequencies which are desirable to be transmitted and received by the analysis system 100.

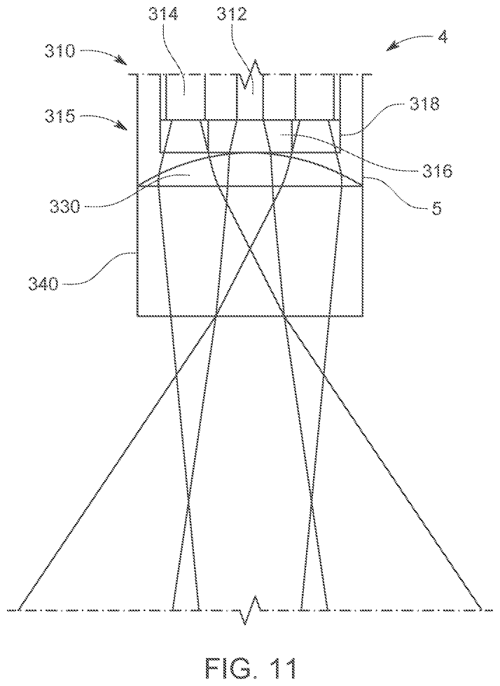

FIG. 11 illustrates another form of the technology in which the probe 4 comprises a transmission window 340. The transmission window 340 may be positioned between the filters 315 (if present) and the subject 500.

The transmission windows 340 may allow for dispersion of the outgoing light from the probe 4 to create a larger spot on the subject 500 than would otherwise be the case. Accordingly, the length of the transmission window may, in different forms, be adjusted to adjust the spot size, and/or allow for lenses with a different focal length.

The transmission windows 340 may be constructed from magnesium fluoride (MgF2), barium fluoride (BaF2), calcium fluoride (CaF2) or quartz.

By way of example, the inventors have determined that, for a spot size of approximately 1 mm, an approximately 2 mm long transmission window 340 may be preferred, while for a spot size of approximately 1.2 mm diameter, an approximately 3 mm long transmission window 340 may be preferred. If lens 330 has a relatively thin construction, the divergence of the outgoing light is largely due to refraction in the transmission window material, which in one example is a magnesium fluoride crystal. Note that the lens 330 may have a width of between approximately 50 μm and approximately 250 μm wide, such as approximately 100 μm, whereas the transmission window 340 may be between approximately 1 mm and approximately 5 mm, such as between approximately 2 mm and approximately 3 mm. In this way, using a longer transmission window 340 allows the outgoing light to expand/diverge more than is possible with conventional probes 4. For example, in conventional laser spot measurement devices, the projected spot size may be approximately 0.6 mm in size.

It may be advantageous to provide a relatively large spot size in some examples of the technology in order to allow for an increased power of stimulus generator 420. For example, a 30 mW stimulus generator (such as a laser) may be used in order to obtain a good signal to noise ratio, while keeping the power density at the surface of the subject 500 to stay within some maximum permitted exposure (MPE) limits for skin.

In forms in which the length of the transmission window 340 is greater, it may be desirable for the radius of curvature of the lens 330 to be selected to also increase the effective focal length (EFL) as should be familiar to those skilled in the art.

### 6.2.2.4. Lens

In some examples of the technology, it may be advantageous for the transmission window to comprise a lens to aid in focusing or defocusing the outgoing light from the probe 4, and incoming light collected by the probe 4. In other examples, the transmission window may be substantially planar, or otherwise substantially perpendicular to the longitudinal axis of the probe 4 so as to provide minimal deflection of the outgoing or incoming light. In yet further examples it may be advantageous to use a transmission window which comprises a substantially planar region and a lensed region.

In the form shown in FIG. 11, the probe 4 comprises a lens 330. The lens 330 may be positioned between the filters 315 (if present) and the transmission window 340. In the illustrated form, the tips of the light guides 310, the filters 315, the lens 330 and the transmission window 340 are positioned immediately adjacent each other.

In some forms of the technology, it may be advantageous for the probe 4 to comprise a lens 330 to aid in focusing or defocusing the outgoing light from the probe 4, and collected light received by the probe 4. The lens 330 may be a convex lens, such as a plano-convex lens. Use of a plano-convex lens may advantageously aid in converting light collected from a spot on the subject 500 to parallel light rays which can be received by the detector, for example through the collecting light guides 314. It may be advantageous in some forms for the illuminating light guide 312 to be substantially aligned perpendicular to the lens 330 along the principle axis of the lens, such that the lens 330 provides minimal deflection of the illuminating light.

The lens 330 may be constructed of any suitable material known to those skilled in the art, including sapphire and diamond, for example. Sapphire and diamond are hard materials and are substantially transparent to light at wavelengths that may be used in exemplary analysis systems 100 according to the present technology. Some exemplary dimensions of a lens 330 are described above.

Further information on components that may be suitable for use in a probe 4 according to certain forms of the technology are described in Australian Patent Application No. 2022903699, the contents of which are herein incorporated by way of reference in their entirety.

### 6.2.2.5. Retaining Structures

The probe 4 may be retained within the housing 210. As shown in FIGS. 3 to 5, 13B-C, 14B-C and 17 to 20, the probe 4 may be wholly contained, or substantially wholly contained within the housing 210. The probe 4 may be retained within the housing 210 so that the tip 5 of the probe 4 is positioned inside the probe tip shield 7, while other, more subject-distal, parts of the probe 4 are contained within the housing body 10. The probe 4 may be oriented with its longitudinal axis substantially parallel to a longitudinal axis of the housing 210.

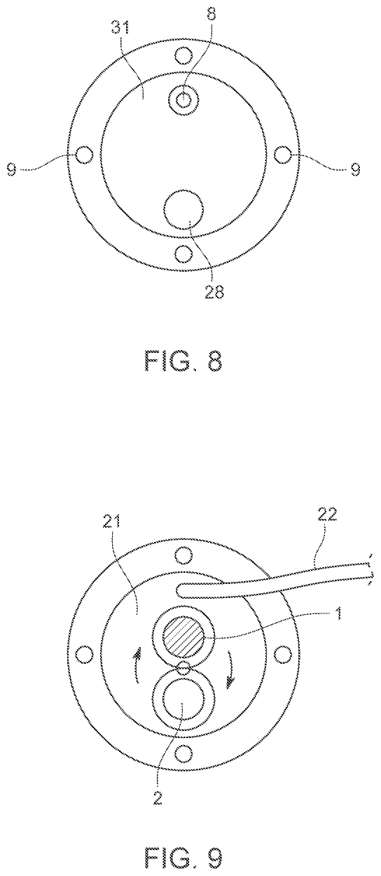

In order to retain the probe 4 within the housing 210, the housing 210 may comprise one or more retaining structures. For example, the housing body 10 may comprise openings at one or both ends (at both ends in the example of FIG. 4) through which respective ends of the probe 4 extend with the openings sized to provide an interference fit with the respective ends of the probe 4 and to retain the probe 4 in position. The opening in the end wall 31 of the housing body 10 that connects to the probe tip shield 7 is labelled as opening 28 in FIGS. 4 and 8. End wall 31 of the housing body 10 is illustrated in FIG. 8. In this exemplary form, the opening 28 is positioned radially offset from the longitudinal central axis of the housing 210. As a result, the opening 220 in the distal end of the probe tip shield 7 may be similarly offset.

In the example of FIG. 4, an additional retaining structure in the form of clamp 19 may be provided inside housing body 10. Clamp 19 extends outwardly from an inner surface of a wall 21 of the housing body 10 and retains a middle section of the probe 4 in position.

### 6.2.3. Retraction Mechanism

In certain forms, the probe assembly 200 comprises a retraction mechanism configured to move the probe 4 between an extended position and a retracted position. FIGS. 4, 5 and 18A illustrate the probe 4 in an exemplary extended position, where the tip 5 of the probe 4 is positioned substantially flush with the end of the housing 210, i.e. the distal end of the probe tip shield 7. In other forms, the tip 5 of the probe 4 may not extend so far out in the extended position, for example in the case of FIGS. 17A, 19A and 20, the tip 5 may be inset from the distal end of the probe tip shield 7 in the extended position. In the retracted position, the tip 5 may be positioned inside the housing 210, for example inside the housing body 10 or inside the probe tip shield 7. More generally, the tip 5 may be positioned further away from the distal end of the probe tip shield 7 in the retracted position compared to the extended position.

In certain forms of the technology, the probe 4 may move longitudinally, i.e. in the direction of its length, between the extended and retracted positions.

Any suitable retraction mechanism may be used in various forms of the technology. In the example shown in FIG. 4, the probe assembly 200 comprises an actuator 18 configured to act on the probe 4 and extend and retract it, for example to move it forward and backward longitudinally. The retraction mechanism may comprise gripping members to grip a section of the probe 4 in order to impart movement on the probe 4 from the actuator 18. The actuator 18 may comprise a motor. The actuator 18 may be mounted to an inner surface of a wall 21 of the housing body 10, for example the side wall, although in other forms the actuator 18 may be positioned elsewhere within the housing body 10.

The parts of probe assembly 200 that function to retain the probe 4 within the housing 210 may also act as guides to help keep the probe 4 in position through the retraction and extension movement.

Being able to retract the probe 4 may enable the user to have a clearer view of the subject 500 than would otherwise be the case if the probe 4 could not be retracted. This may enable the user to position the probe 4 more accurately for analysis of the subject 500. It may also enable an image of the subject 500 to be captured for storage and record keeping of the analysis, or for later analysis (using the image capture mechanism that will be described later). Being able to vary the position of the probe 4 in the extended position may additionally, or alternatively, enable the probe 4 to accommodate subjects 500 that are not entirely flush with the surrounding regions, or reduce the discomfort caused by pressure of the probe on a skin lesion.

### 6.2.3.1. Pressure Control Mechanism

In certain forms, the retraction mechanism may comprise a pressure control mechanism for controlling a pressure exerted by the tip 5 of the probe 4 on the skin region 510 when the probe 4 is in the extended position. The pressure control mechanism may assist in avoiding the probe 4 exerting an uncomfortable, or harmful, level of force on the skin region 510 during the analysis process. It may also help provide consistency in the level of force exerted by the probe 4 on the skin region 510 during the analysis process, which may improve the consistency in the spectral analysis results. Controlling the amount of pressure exerted on the subject may be useful because different parts of the body and different types of skin lesion may protrude from the surrounding region of the subject's skin by differing amounts. Different forms of the technology may use different forms of pressure control mechanism and a few examples are provided below.

In some forms, for example as illustrated in FIG. 4, the pressure control mechanism may comprise an actuator controller 25 to control the actuator 18 to control the retraction and extension of the probe 4, for example to control the speed of retraction and extension, and/or to control the limits of movement of the probe 4 in the retracted and extended positions, for example to control the retracted and extended positions to be as described above. The limit of movement in the extended position may be particularly useful for controlling the amount of pressure exerted by the tip 5 on the skin region 510.

The actuator controller 25 may be configured to control the limit of movement in the extended position based on one or more of a variety of factors, including feedback from one or more other parts of the probe assembly 200 (such as described below) and manual control by a user. The actuator controller 25 may be configured such that the position of the tip 5 in the extended position may differ with each use of the probe assembly 200. The actuator controller 25 may be mounted to an inner surface of a wall 21 of the housing body 10, for example the side wall, although in other forms the actuator controller 25 may be positioned elsewhere within the housing body 10.

In some forms, the actuator controller 25 may be operated by direct control by a user. A user interface may be provided, for example on the side of the probe assembly 200, to allow the user to directly control the movement of the probe 4. The user interface may comprise any suitable input mechanism, e.g. buttons, dial, scroll wheel, etc.

In some forms, the pressure control mechanism may comprise a pressure sensor configured to sense pressure exerted on the probe 4, particularly pressure exerted on the probe 4 in the longitudinal direction that acts to resist further extension of the probe 4, such as the pressure that is exerted on the probe 4 by the skin region 510 during use. In some forms, the probe assembly 200 may comprise an output display configured to display the pressure sensed by the pressure sensor, or an indication of the level of the pressure, to a user. The user may be able to manually control the extension of the probe 4 in order to achieve the desired pressure. Alternatively, the actuator controller 25 may be configured to receive signals from the pressure sensor indicative of the sensed pressure and to control movement of the probe 4 based on the sensed pressure. For example, the actuator controller 25 may be configured to prevent further movement of the probe in the forward (extending) direction if the sensed pressure exceeds a predetermined threshold. Alternatively, the actuator controller 25 may be configured to permit further extension by a predetermined amount once the sensed pressure on the probe 4 exceeds a predetermined threshold. In an example of how this operates in practice, if a part of the subject 500 extends into the opening at the distal end of the probe tip shield 7, the probe 4 may contact the subject 500 before it arrives at the fully extended position. In that scenario, the actuator controller 25 may act to stop extension of the probe 4 on detection of a sensed level of pressure by the pressure sensor that indicates contact between the probe 4 and the subject 500. This may make the probe assembly 200 more comfortable to use for the patient, as excessive pressure on some skin lesions may be painful or harmful. Any suitable form of pressure sensor, or combination of pressure sensors, may be used. In some forms, the pressure sensor comprises one or more pressure transducers, switches and/or touch sensors, e.g. conductive sensors or inductive sensors.

In the form of the technology illustrated in FIG. 5, the pressure control mechanism comprises a spring 230 positioned to act between the probe 4 and the housing 210, for example the housing body 10. For example, the spring 230 may take the form of a helical coil mounted around a section of the probe 4.

The spring may act against a surface of the housing body 10, for example by being mounted inside a spring housing 232. The outer surface of the probe 4 may comprise a protrusion or indentation forming another surface that the spring 230 may act against. Alternatively, the spring 230 may be sufficiently tightly wound around the probe 4 so as to grip the probe 4 through friction. The spring 230 may be structured to allow the probe 4 to retract when a force pushes the tip 5 of the probe 4 inwardly, and then to return the probe 4 to its rest position when the force is removed. The rest position of the probe 4 may be where the tip 5 of the probe 4 is positioned substantially flush with the end of the housing 210, i.e. the distal end of the probe tip shield 7. In operation, the spring may enable the probe 4 to accommodate subjects 500 that are not entirely flush with the surrounding regions.

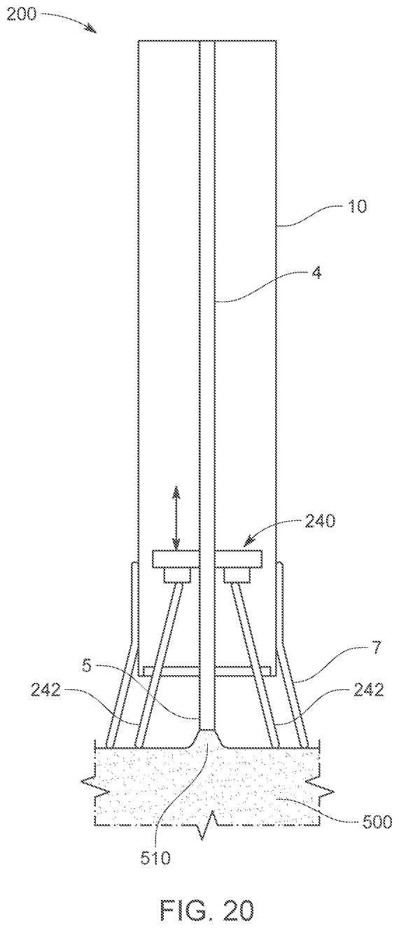

In some forms of the technology, the pressure control mechanism comprises one or more components of the probe assembly 200 that are configured, in use, to act on the subject's body and physically push or otherwise manoeuvre a part of the subject's body in order to alter the pressure exerted by the probe 4 on the body. These one or more components are termed a “prod assembly” in the ensuing description because they act to prod on the subject's body. For example, the probe assembly 200 shown in FIG. 20 comprises a prod assembly 240 configured to urge one or more prod members 242, which may be comprised as part of the prod assembly 240, against a region of the subject's body proximate the skin region 510. The prod assembly 240 may, in some forms be housed within the housing body 10. In particular, the prod member 242 may be positioned inside the probe tip shield 7 so that the prod member 242 is able to act on the subject's body while the skin region 510 is shielded from ambient light. The prod assembly 240 may further comprise a prod actuator (not shown) configured to move the prod member 242, for example to retract and extend the prod member 242 longitudinally relative to the probe assembly 200. The prod actuator may be controlled manually by a user, through a suitable control interface, or the prod actuator may receive feedback from a pressure sensor in order to achieve a desired level of pressure exerted by the probe 4 on the skin region 510, optionally in conjunction with the actuator controller 25. In the form of FIG. 20, the prod members 242 comprise two rods projecting outwardly from the housing body inside the probe tip shield 7. The two rods may be positioned diametrically opposite each other. The distal ends of the rods may be configured to be spaced apart to allow skin region 510 to be positioned in between the distal ends in use. The rods may be angled away from each other towards their distal ends. In other forms, the prod member(s) 242 may take other forms, for example plates, pads, prods with curved (e.g. ring-shaped) distal ends, or the like. The prod assembly 240 may, in some forms, be useful to help to move certain body features into a position where they do not interfere with the operation of the probe assembly 200. For example, the prod member 242 may be used to fold the ear over to enable the probe 5 to analyse a skin region 510 situated behind the ear. The prod member 242 may be formed with a shape that facilitates such a function.

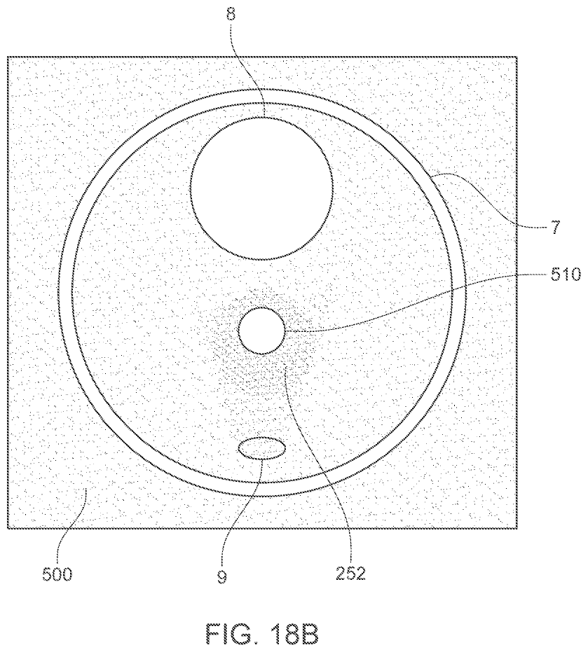

In some forms, the pressure control mechanism comprises an imaging assembly 250. The imaging assembly 250 may be configured to determine when the tip 5 is in contact with the skin region 510. For example, in the forms shown in FIGS. 17A-B and 18A-B, the imaging assembly 250 may comprise a camera 8 positioned to image the skin region 510 when the probe assembly 200 is in use, i.e. the camera 8 may be positioned such that the field of view of the camera 8 includes the tip 5 of the probe 4 when in the extended position, and an area around the tip 5. A further discussion on suitable positioning of a camera 8 is provided later. The imaging assembly 250 may further comprise one or more light sources 9 to illuminate the subject 500 when the subject is positioned in, or directly in front of, the distal end of the probe tip shield 7. Images captured by the camera 8 may be sent to a processor, for example processor 440 (described later), for analysis to determine when the tip 5 comes into contact with the skin region 510, and optionally an amount of pressure exerted by the tip 5 on the skin region 510. Any of a number of techniques to analyse the images captured by the camera 8 may be used to determine if the tip 5 is in contact with the skin region 510, and optionally if the skin region 510 is depressed by the tip 5, for example by analysing the position of the shadow of the tip 5 relative to the tip 5 itself and/or analysing shadows 252 created by depressions in the skin region 510 and/or surrounding regions of skin, as shown in FIGS. 18A and 18B. In such forms, the actuator controller 25 may be configured to limit movement of the probe 4 based on the determination of the imaging assembly. For example, the processor 440 may be configured to send a control signal to the actuator controller 25 once it has been determined that a desired level of pressure exerted by the tip 5 on the skin region 510 has been reached so that the actuator controller 25 may stop movement of the probe 4. In other forms, a user may view the images captured by camera 8 to manually control the extension of the probe 4 to the desired position.

In certain forms, the pressure control mechanism comprises a mapping assembly 260. The mapping assembly 260 may be configured to determine a shape of the skin region 510, and optionally a region of the subject 500 surrounding the skin region 510. For example, in the form shown in FIGS. 18A-B, the mapping assembly 260 may comprise a camera 8 positioned to image the skin region 510 when the probe assembly 200 is in use, i.e. the camera 8 may be positioned such that the field of view of the camera 8 includes the tip 5 of the probe 4 when in the extended position, and an area around the tip 5. The mapping assembly 260 may further comprise a plurality of light sources 9 to illuminate the subject 500 when the subject is positioned in, or directly in front of, the distal end of the probe tip shield 7. In the form of FIGS. 18A-B, the light sources 9 generate light of different colours (for example red, green and blue), which may enable images captured by the camera 8 to be sent to a processor, for example processor 440, for analysis to determine the shape of the skin region 510, and optionally the region of the subject 500 surrounding the skin region 510. Conventional stereoscopic mapping processes may be used to perform this analysis. Once the shape of the skin region 510 has been determined, the actuator controller 25 may be configured to limit movement of the probe 4 based on the determination of the mapping assembly 260. For example, the processor may send signals to the actuator controller 25 to achieve the amount of extension of the probe 4 that is determined to be appropriate to achieve the desired level of pressure exerted by the tip 5 on the skin region 510.

In some forms, the pressure of the tip 5 on the skin region 510 may be determined by operating the probe 5 and using the processor 440 to analyse the spectral signal received by the detector 430 to determine whether there is contact between the tip 5 and the skin region 510. During this process, the stimulus generated by the stimulus generator 420 may be at a relatively low level so that, if the probe 4 is not correctly positioned, the subject is not harmed. The nature of spectra that indicate contact or lack of contact with a subject's skin may be able to be determined through experiment, and the processor 440 subsequently programmed to identify the nature of the spectrum received, e.g. through an AI training process.

### 6.2.4. Viewing Assembly

Probe assemblies according to certain forms of the technology may comprise a viewing assembly, which enables a user to view the subject 500 that is analysed, or will be analysed by the probe 4 in use. In the form of technology shown in FIGS. 3 to 5, 13 to 15, 17, 18 and 22C, the viewing assembly comprises a camera 8 and a screen 16. The camera 8 is positioned so that the location of the subject 500 when the probe assembly 4 is being used is within its field of view so that the camera 8 captures images of the subject. Since, in use, the probe assembly 4 is positioned so that the subject 500 is located in, or directly in front of, the distal end of the probe tip shield 7, the camera 8 may be positioned so that the opening at the distal end of the probe tip shield 7, or part thereof, is within its field of view. The positioning of the camera 8 may also mean that the tip 5 of the probe 4 is within its field of view when the probe 4 is in an extended position so that the camera 8 may view the skin region 510 when the distal end of the probe tip shield 7 abuts a region of the subject's body surrounding the skin region 510.

In the illustrated forms, the camera 8 is positioned inside the housing 210. Since the housing may be formed to be opaque, and since the probe assembly 200 is positioned in use to abut subject 500 and thereby prevent light entering into the probe tip shield 7 through the opening at its distal end, no ambient light may enter the camera's field of view when the camera is used to capture images, and this means that the lighting of the field of view can be controlled. In the examples of FIGS. 4, 5 and 8, the camera 8 is mounted in an end wall 31 of the housing body 10. For example, the camera 8 may be positioned adjacent the opening 28 in the end wall 31. In other forms, the camera may be mounted on an end wall 31 of the housing body 10, or on a side wall of the probe tip shield 7.

Since the probe assembly 200 may be configured so that no ambient light enters the camera's field of view when in use, the viewing assembly may further comprise one or more light sources 9 to illuminate the subject 500 when the subject is positioned in, or directly in front of, the distal end of the probe tip shield 7. In the forms shown in FIGS. 4 and 8, 17 and 18, the one or more light sources 9 are mounted on an end wall 31 of the housing body 10. For example, there may be four light sources 9 each positioned proximate the outer circumference of the end wall 31 of the housing body 10. In other forms, the one or more light sources 9 may additionally or alternatively be mounted on a side wall of the probe tip shield 7. In some forms, the one or more light sources 9 may additionally or alternatively be mounted on the camera 8. The light sources may be LEDs or any suitable source of light. A user interface control may be provided to enable the light sources to be turned on and off, for example buttons 12 and 13, which may be positioned on an outer surface of the housing 210. In other forms, the light sources may be configured to automatically be activated when the camera is activated, and subsequently automatically de-activated. Buttons 12 and 13 may be operatively connected to the light sources 9, for example through wires 24. The light sources may be configured to generate light at wavelengths that will not interfere with the nature of the light used to illuminate the subject 500 for analysis, and the nature of the light collected from the subject for analysis. For example, in some forms, the light sources may be configured to generate optical white light and/or optical blue light.

Screen 16 may be configured to display images from the camera 8 to the user. Any suitable type of screen 16 may be used. As shown in FIGS. 3 to 5, 13 to 15, 17, 18 and 22C, the screen 16 may be positioned outside housing 210 for convenient viewing by the user. Alternatively, the screen 16 may form part of a wall of the housing 210, i.e. the wall of the housing may comprise the screen 16. In the illustrated forms, the screen 16 is mounted on a panel 17. The panel 17 may be mounted to the housing body 10, for example movably (e.g. pivotally) mounted so that the user may alter the position or orientation of the screen 16 to achieve convenient viewing.

The camera 8 may be configured to capture, and the screen 16 may be configured to display, still images and/or video images.

The viewing assembly may be configured such that the screen 16 displays images from the camera 8 to the user with one or more alignment markings added to the image to indicate the position of the tip 5 of the probe 4, or a future position of the tip when the probe 4 is in the extended position, to assist the user to position the probe assembly 200 correctly in order to analyse the intended skin region.

FIGS. 4, 5 and 9 illustrate one or more wires 22 that connect to the camera 8. The wires may supply power and control signals to the camera 8, and convey signals indicative of captured images from the camera 8. The one or more wires 22 may be connected to screen 16 and to a power supply, which may be a power supply 23 comprised as part of the probe assembly 200 or a power supply 470 located remotely from the probe assembly 200, for example in portable unit 400. As shown in FIG. 9, in some forms, the wires 22 may pass through an opening in end wall 31 in order to connect to the camera 8.

The viewing assembly may further comprise a user interface control to enable a user to activate the camera 8, for example button 14. Button 14 may be operatively connected to the camera 8, for example through wires 24. Button 14 may be positioned on an outer surface of the housing 210.

In some forms, communication of data to and from the camera 8, and/or control of the camera 8, may be wireless, for example through one or more appropriate wireless communication protocols such as Bluetooth, WiFi, NFC, RF, etc.

In some forms of the technology, the images from the camera 8 may be stored in a memory comprised as part of the probe assembly 200. This storage of camera images may occur additionally or alternatively to the communication of images to the screen 16 for display to the user. In some forms of the technology, the images from the camera 8 may be communicated to a location remote from the probe assembly 200. This remote communication of camera images may occur additionally or alternatively to the communication of images to the screen 16 for display to the user and/or to storage of the camera images in a memory on the probe assembly. The camera images may be displayed to a user at the remote location or stored in a memory for later use, e.g. display or analysis. The remote location may, in some forms, be processor 440, which may be comprised as part of portable unit 400. In such forms, the camera images may be displayed on output device 450 comprised as part of the portable unit 400. In other forms, the remote location may be any other data receiving device, for example a computer, smartphone or other portable communication device. Communication of the camera images to a location remote from the probe assembly 200 may occur through wired or wireless communication. For example, wired communication may occur through wires 32 housed within conduit 33. Such wires may connected to processor 440, for example in portable unit 400. Alternatively (or, in some forms, additionally), the camera images may be communicated through one or more appropriate wireless communication protocols such as Bluetooth, WiFi, NFC, RF, etc.

The images captured by the camera 8 may be used for any one or more of a number of purposes, including but not limited to any one or more of: real-time viewing by a user to guide placement of the probe assembly 200 (for example to ensure alignment of the tip 5 of the probe 4 with the skin region 510 to be analysed); subsequent analysis of the image by processor 440 for analysis of the visual appearance of the skin region 510; storing an image of the skin region 510 in a memory (e.g. a database) alongside the spectral data of the skin region; and detecting ambient light inside the probe tip shield 7 to determine whether the probe assembly 200 is appropriately positioned for use.

In another form of the technology, the viewing assembly may comprise a window in the wall of the housing 210 which permits the user to view the subject 500 through the window when the probe assembly 200 is correctly positioned for use. The window may be formed in a wall of the probe tip shield 7 or housing body 10. The window may be formed as an opening in the wall, or the window may comprise a substantially transparent member positioned in an opening in the wall. The substantially transparent member may be a transparent panel or a lens, for example. In such forms, the viewing assembly may comprise an opaque closure member, for example a pivoting or sliding flap, able to cover the window when the probe 4 is activated in order to substantially prevent ambient light shining on the subject 500 when the probe 4 is in use. In some forms, the window may be configured with alignment markings, e.g. cross-hairs, to indicate a position of the tip 5 of the probe 4, or a future position of the tip when the probe 4 is in the extended position, to assist the user to position the probe assembly 200 correctly in order to analyse the intended skin region.

### 6.2.5. Calibration Member and Mechanism

In certain forms of the technology, the probe assembly 200 may comprise a calibration mechanism. The calibration mechanism may be configured to enable calibration of the analysis system 100, i.e. to ensure that the measured parameters of the detected spectrum, e.g. wavelengths and/or frequencies, substantially equal the true values of these parameters (which may be referred to as wavenumber calibration). In some forms, the calibration mechanism operates by detecting the spectrum of a material with a known spectrum, checking the detected spectrum against the known spectrum and, if necessary, altering the way in which the spectrum is analysed to compensate for any differences. In the relevant forms of the technology, the calibration mechanism in the probe assembly 200 comprises a mechanism that enables the probe 4 to illuminate and collect light from a material having a known spectrum when exposed to certain type of light from the probe 4.

In exemplary forms of the technology, for example those illustrated in FIGS. 4 and 5, the probe assembly 200 comprises a calibration member 1 formed from a material that produces a known spectrum when exposed to light of a specific wavelength, or wavelength range. For example, in some forms, the calibration member 1 may be formed of silicon or calcite. Other suitable materials may alternatively be used.

The calibration mechanism may be configured to alter the relative position of the calibration member 1 and the probe 4. This may be achieved by moving the calibration member 1 only, moving the probe 4 only, or by moving both the calibration member 1 and the probe 4. The relative position of these two components may be altered to move them between two configurations, which will be referred to as a “calibration configuration” and a “subject configuration” in order to help distinguish the two configurations for the purposes of this description. In the calibration configuration, the probe 4 is configured to analyse the calibration member 1 and thereby perform calibration of the analysis system 100. In the subject configuration, the probe 4 is configured to analyse the subject 500. In the calibration configuration, the calibration member 1 may be positioned in front of probe 4, for example directly in front of the tip 5 of probe 4. In the subject configuration, the calibration member 1 may not be positioned in front of probe 4. Instead, the subject 500 may be positioned in front of probe 4, for example directly in front of the tip 5 of probe 4.

The calibration mechanism may comprise an actuator 3 for moving the calibration member 1 between positions in the calibration configuration and subject configuration. Another actuator may also move the probe 4 between positions in the calibration configuration and subject configuration, and this other actuator may, in some forms, be the retraction actuator 18 as will be explained below. That is, the calibration mechanism may comprise the retraction mechanism so that, in the calibration configuration, the probe 4 is in the retracted position, and, in the subject configuration, the probe 4 is in the extended position.

The exemplary calibration mechanism of the form illustrated in FIGS. 4 and 9 will now be described. In this form, the calibration member 1 is movably mounted inside the housing 210. For example, the calibration member 1 may be pivotably mounted inside the housing body 10, for example to an inner surface of one of the walls of the housing body 10. In the illustrated form, calibration member 1 is pivotably mounted to a pivot rod which extends outwardly from an inner surface of the end wall 31 in a direction substantially parallel to the longitudinal axis of the housing 210. In some forms, the calibration member 1 is mounted on a carrier, which is itself pivotably mounted to the rod. The actuator 3 is configured to move calibration member 1 between its positions in the calibration and subject configurations, for example by rotating it around the pivot rod. Actuator 3 may be located in any suitable position but, in the example of FIG. 4, is shown as being mounted to the end wall 31.

The probe assembly 200 may be configured so that the probe 4 is positioned adjacent to the position of the pivot rod, as shown in FIG. 4. FIG. 4 illustrates the subject configuration, in which the calibration member 1 is positioned out of alignment with the axis of probe 4, and the probe 4 extends through the opening 28 in end wall 31 into the probe tip shield 7. In some forms, for example as illustrated in FIG. 4, the probe assembly may additionally comprise an additional guide member 2 through which the probe 4 may extend, although this may not be present in other forms. In FIG. 4, the guide member 2 is a ring through which the probe 4 may extend. The ring is also pivotably mounted to the pivot rod but in a position opposite to the mounting of the calibration member 1. In the subject configuration, the ring is axially aligned with opening 28 in end wall 31. Guide member 2 may help prevent actuator 3 being actuated when the probe 4 is extended and causing the calibration member 1 to impact the side of the probe 4, which may cause damage.

In the calibration configuration, the calibration member 1 is positioned in axial alignment with the longitudinal axis of the probe 4. The actuator 3 may cause calibration member 1 to pivot around the pivot rod to reach this position. In the form shown in FIG. 4, the presence of probe 4 in the extended position prevents this being achieved and so first probe 4 must be retracted by actuator 18 so that the tip 5 of the probe 4 is retracted backwards past the longitudinal position of the calibration member 1. In some forms, the actuation of actuators 3 and 18 may be controlled together so that actuator 3 only begins to move calibration member 1 once the probe 4 has been retracted suitably far, so actuators 3 and 18 may be connected, for example with wires 29. Where a guide member 2 is present, this means that the probe 4 is retracted out of guide member 2 and no longer passes through the guide member. This allows the calibration member 1 to be able to rotate into alignment with the probe 4, and guide member 2 may pivot out of alignment. In the calibration configuration, the probe 4 may be positioned so that the calibration member 1 is positioned in front of, for example directly in front of, the tip 5 of the probe 4. This allows activation of the analysis system 100 in order to spectrally analyse the calibration member 1, and consequently allows calibration of the analysis system 100., for example by the processor 440.

Other mechanisms for calibrating the analysis system 100 are described below in the section describing the portable unit.

### 6.2.6. Cleaning Mechanism

In certain forms of the technology, the probe assembly 200 may comprise a mechanism for cleaning the tip 5 of the probe 4. It may be useful to clean the tip 5 in between uses, for example when used with a different subject, in order to avoid contamination of one subject with matter from another.

In some forms, a cleaning member may be positioned inside the housing 210 in a location where, when the probe 4 is retracted, the tip 5 comes into contact with the cleaning member. The cleaning member may be treated with a cleaning agent, for example a disinfectant. In some forms, the cleaning member may comprise an absorbent material, e.g. a sponge, soaked with a fluid cleaning agent.

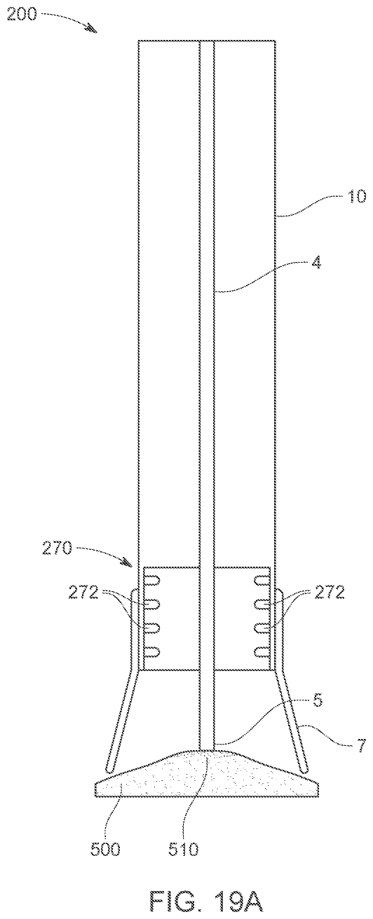

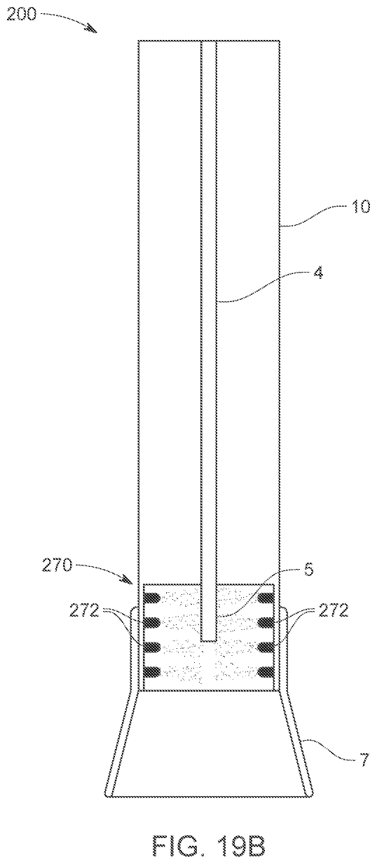

Another form of cleaning mechanism 270 is illustrated in FIGS. 19A and 19B. In this form, the cleaning mechanism 270 comprises one or more ultra-violet light sources 272 positioned inside the housing 210 and configured to illuminate the tip 5 of the probe 4 with ultra-violet light when the probe 4 is in the retracted position. For example, a plurality of UV light sources 272 may be positioned on an interior surface of a wall of the housing body 10 and configured to project UV light radially inwardly towards the probe 4. The UV light sources 272 may be positioned longitudinally along the housing 210 approximately level with the longitudinal position of the tip 5 when the probe 4 is retracted. The UV lights sources 272 may be able to be activated with a suitable control mechanism in order to expose the tip 5 with UV light of a sufficient intensity and for a sufficient duration to substantially sterilise the tip 5. For example, the UV light sources 272 may be activated manually following a user activating a control, or automatically when the probe 4 returns to a retracted position.

### 6.2.7. Safety Mechanism

Operation of the probe assembly 200 has the potential to cause harm to the subject if used in an unintended manner. For example, if light of a certain nature (e.g. of certain wavelengths or with high intensity) is projected at certain parts of the body, harm may occur. Of particular concern is the human eye. Consequently, the probe assembly 200 may incorporate one or more features that mitigate against the risk of unintentionally shining light into the eye. Some examples of such features will now be described.

In some forms, the probe assembly 200 may comprise one or more pressure sensors, such as has been described above in relation to the pressure control mechanism. Feedback from the pressure sensors may be used as a safety mechanism and the stimulus generator 420 may only be able to be activated if that feedback meets certain criteria that have been set for being determinative of the probe assembly 200 being in a position that is safe to use. The pressure sensors that are comprised as part of the safety mechanism now being described may be the same as, or in addition to, the pressure sensors that are comprised as part of the pressure control mechanism.

In some forms, the probe assembly 200 may comprise a plurality of pressure sensors positioned at the distal end of the probe tip shield 7 and configured to measure pressure on the probe tip shield 7. The analysis system 100 may be configured so that the stimulus generator 420 may not be activated unless all of the pressure sensors are sensed as being in contact with something. In some forms, each pressure sensor may need to sense a pressure reading above a predetermined threshold for activation of the stimulus generator 420 to occur. In some forms, the pressure sensors may comprise pressure transducers, conductive sensors and/or inductive sensors. In some forms, one or more of the pressure sensors may each comprise a switch, e.g. a mechanical switch or a pressure switch and the analysis system 100 may be configured so that the stimulus generator 420 may not be activated unless each of the switches is closed. In some forms, the probe assembly 200 may be configured to sense contact of the tip 5 of the probe 4 with an object, for example using the pressure sensor(s) of the pressure control mechanism described earlier. The analysis system 100 may be configured so that the stimulus generator 420 may not be activated unless all of the pressure sensors around the probe tip shield 7 and the pressure sensor sensing contact of the tip 5 meet certain criteria, e.g. meet or exceed certain threshold values.

In other forms, the actuator controller 25 may be configured so that the probe 4 may not be extended unless certain criteria from the safety mechanism are met, and the stimulus generator 420 may not be able to activated unless the probe 4 is extended.

In some forms, the safety mechanism may comprise a suction mechanism configured to apply a negative pressure to the volume inside the probe tip shield 7. The safety mechanism may further comprise one or more air pressure sensors, e.g. one positioned to measure the air pressure inside the housing 210 (e.g. inside the probe tip shield 7) and one positioned to measure the ambient air pressure (e.g. the sensor may be located on an outer surface of the housing 210). If a pressure difference indicates that the tissue is being sucked up into the probe tip shield 7, this may indicate that the tissue is suitable for analysis, in which case the stimulus generator 420 may be able to be activated.

In some forms, the safety mechanism may involve analysis of images captured by the camera 8. If this analysis determines that the object in front of the probe 4 is unsuitable for analysis, then the stimulus generator 420 may not be activated or, vice versa, the analysis system 100 may be configured not to activate the stimulus generator 420 until an object considered safe to analyse is identified in the images captured by the camera 8. In certain forms, object identification algorithms may be applied to the images. For example, a machine learning model trained to recognise eyes may be applied to the images and the stimulus generator 420 may not be able to be activated following a determination that an eye is in the field of view of the camera 8.

In certain forms, the safety mechanism may involve the manner in which the stimulus generator 420 is activated. For example, the stimulus generator 420 may be configured to initially generate a stimulus signal at level of intensity and wavelength that is not damaging to the human eye but is at a level sufficient to trigger an aversion response to help mitigate against the eye being in front of the probe 4 when the full analysis stimulus is generated.

### 6.2.8. Other Features of Probe Assembly

The probe assembly 200 may comprise one or more user interfaces. The user interface enables a user to control certain feature of the probe assembly 200, for example activating and/or de-activating certain mechanisms or functions of the probe assembly 200. Certain examples of user interface have already been described, for example the buttons 12, 13 and 14 for activating/de-activating the camera 8 and light sources 9. In addition, a user interface control, optionally in the form of a button 11, may be provided to activate the probe 4 in order to analyse the subject 500. In addition, a further user interface control, optionally in the form of a button 30 or switch, may be provided to turn the probe assembly 200 on and off, for example to selectively supply power to the probe assembly 200.

The probe assembly 200 may comprise any suitable types of user interface. For example, while FIG. 4 illustrates a plurality of buttons, other types of control interface may be provided such as: switches, dials, touch pads, touch screen displays. These types of interfaces may be provided in any combination. In other forms, the control of the probe assembly 200 may be achieved remotely, for example through a remote control device connected to the probe assembly 200 through a wired or wireless connection. In some forms, the remote control device may be a dedicated handheld controller or any suitable device, such as a computer, laptop, tablet or smartphone, for example.

It has already been explained that probe assembly 200 may comprise a power supply 23, as shown in FIG. 4, while in other forms a power supply 470 may be located remotely from the probe assembly 200, for example in portable unit 400, and connected to the probe assembly 200 through power-carrying wires. In some forms, the power supply 23 may comprise a rechargeable battery, and the probe assembly 200 may comprise a port 26 connected to the battery, and located in a wall 21 of the housing body 10, through which the battery may be recharged. The port 26 may be a USB port, for example, or any other suitable type of port.

In some forms, the probe assembly 200 may comprise a controller, for example a Raspberry Pi or other microcontroller. The controller may be operatively connected to any one or more of the features of probe assembly 200 to control their operation, for example through wires 15.

In some forms, the probe assembly may comprise an alignment light source for projecting light to facilitate alignment of the probe 4 with the intended skin region 510 to be analysed. For example, the alignment light source may be configured to project a ray of light in a direction so that, the ray projects a spot of light onto an object placed closely in front of the distal end of the probe tip shield 7, indicating where the tip 5 of the probe 4 will be positioned when the probe 4 is in an extended position. This spot of light may be easily seen by a user, and the light source may be able to be turned off when the probe assembly 200 is correctly positioned. In some forms, the alignment light source may be the stimulus generator 420 (described below) generating light at a relatively low-level (compared to when the stimulus generator 420 generates light for the purpose of spectral analysis) at a visible wavelength and the light may be projected onto the subject 500 by one or more of the light guides 310. In other forms, a separate light source of the light guides may be provided.

### 6.3. Conduit

In some forms of the technology, the analysis system 100 may comprise a conduit 33. The conduit 33 may be configured to convey any one or more of the following between the probe assembly 200 and other components of the analysis system 100: light for illuminating the subject 500; collected light from the subject 500; power; and control signals.

For example, the conduit may comprise a plurality of light guides 20. The light guides 20 may be physical extensions of the light guides (both the illuminating and collecting light guides of the probe 4), or may be otherwise optically connected (i.e. configured to communicate light) to the light guides of the probe 4. The light guides 20 may be fibre optic cables, for example. At the end of the conduit distal from the probe assembly 200, the illuminating light guides 20 may be optically connected to the stimulus generator 420 and the collecting light guides 20 may be optically connected to the detector 430.

In addition, the conduit 33 may comprise wires 32. The wires 32 may be connected to processor 440, for example in portable unit 400, to convey control signals to/from the probe assembly 200. In some forms, the wires 32 may be connected to the power supply 470 to supply power to the probe assembly 200.

The conduit 33 may comprise a sheath to contain and protect the light guides and wires of the conduit 33. The sheath may be a sleeve, for example formed from plastic. In some forms, the conduit 33 may be between 500 and 5000 mm in length.

### 6.4. Stimulus Generator

In certain forms of the technology, the analysis system 100 comprises a stimulus generator 420 configured to generate light for exposure of the subject 500 with said light. In certain forms, the stimulus generator 420 may comprise one or more lasers, for example laser diodes. In one example the light is generated within the spectrum of near infrared light, for example wavelengths of approximately 700 to 1400 nm, for example approximately 800 to 850 nm, for example approximately 830 nm. In other examples, light of another wavelength(s) may be used.

In some examples of the technology, the stimulus generator 420 may be configured to generate a plurality of stimulus signals, such as light at a plurality of different frequencies/wavelengths. In these examples the stimulus generator 420 may be configured to generate the stimulus sources simultaneously, while in other examples, the stimulus generator 420 may be configured to generate each of the stimulus sources sequentially, such that only a single stimulus is present at any one time. In some forms, the stimulus generator 420 may comprise a plurality of light sources, each configured to generate light at a different wavelength, while in other forms, the stimulus generator 420 may comprise a single light source configured to generate light at multiple wavelengths.

In certain forms, the stimulus generator 420 may comprise a modulator configured to modulate the light source (e.g. laser) so that the light source is switched on and off sequentially. For example, the modulator may control the light source to pulse at a set frequency (i.e. pulse modulation). The effect of this modulation may be to shorten the total exposure time of the subject 500 to radiation. This may reduce the power density of radiation received at the subject 500 (compared to a stimulus generator that does not pulse) while enabling the use of enough laser power to obtain a good signal-to-noise ratio. In the case where the subject 500 is a patient's skin, modulation may assist in causing the radiation exposure to remain within the exposure range that may be safe for a patient to experience, for example as set by health authorities. In addition, it may enable measurement of a larger volume of a skin lesion, thus also giving a better signal-to-noise ratio.

The stimulus generator 420 may be configured to be activated for a predetermined amount of time, which in some exemplary forms may be 10 to 30 seconds. The analysis system 100 may enable a user to select the activation time through a user interface, or the processor 440 may determine the activation time based on one or more parameters selected by the user.

The stimulus generator 420 may be optically connected to conduit 33 so that the light generated by the stimulus generator 420 is conveyed by the illuminating light guides in the conduit for exposure to the subject 500 by the probe 4.

### 6.5. Detector

The analysis system 100 of certain forms of the technology may comprise one or more detectors 430, configured to receive collected light from the subject 500. The detector 430 may be optically connected to the conduit 33 and the collected light received by the detector 430 is received from the conduit 33, e.g. the collecting light guides 314 provide light to the detector 430. The detector 430 may generate a signal indicative of the collected light, for example by converting received stimulus (light) into electronic readings which can be processed to determine information about the received light.

Suitable types of detector and their techniques of operation should be familiar to those skilled in the art, and therefore we do not discuss these in detail here for sake of brevity, but these should be understood to include detectors such as gratings (such as a diffraction grating), charge coupled device (CCD) detectors, and linear arrays, for example. In some forms, the detector 430 is configured to detect the received light as a Raman spectrum.

### 6.6. Processor

In certain forms of the technology, for example the forms shown in FIGS. 1 and 2, the analysis system 100 may comprise at least one processor 440. The processor 440 is configured to analyse the signal indicative of the collected light from the subject 500, i.e. the signal generated by the detector 430. In certain forms, the processor 440 performs spectral analysis on the signals. For example, the processor 440 may be configured to perform any one or more of: Raman spectroscopy; infrared spectroscopy, near infrared spectroscopy, ultraviolet and visible spectroscopy. The type of spectroscopic analysis may depend on the part of the electromagnetic spectrum used to illuminate the subject 500, and may depend on the nature of the subject 500.

In some examples of the technology, the processor 440 may be an application specific integrated circuit (ASIC), microprocessor, computer processor, or any other suitable processor known to those skilled in the art. The processor 440 may be dedicated to the spectral analysis performed by the analysis system 100. In other forms, the processor 440 may be a processor in a general-purpose computing device, for example a server, PC, laptop, tablet or smartphone, for example. In some forms, the processor 440 may comprise a plurality of individual processor devices that collectively act to analyse the signal. The plurality of processors may be formed from multiple physical processing devices, which may be physically connected or may be physically separated and configured to communicate with each other remotely, for example over the Internet or any other suitable network infrastructure.

In some forms, the analysis system 100 may further comprise a memory and the processor 440 may be configured to record or store information determined as a result of, or as part of, the spectral analysis on the memory. The memory may take the form of a hard drive, solid state drive or removable storage device. In some forms, the memory may comprise a plurality of individual data stores, which may be physically connected or may be physically separated and connected remotely, for example a distributed memory system. In some forms, the memory and processor 440 may be comprised as part of the same computing device, for example a PC, laptop, tablet or smartphone.

### 6.6.1. Spectral Analysis

In certain forms, the processor 440 may be configured to filter the received signal indicative of the collected light from the subject 500. For example, the processor 440 may apply a bandpass filter to the received signal. In other forms, the bandpass filter may be a separate component that filters the signal before analysis by the processor 440. The filter may be configured to filter wavelengths not required for the particular type of analysis being carried out by the analysis system 100. In some forms, the processor 440 may be configured to process the received signal in order to remove etaloning effects, fluorescent background signals and/or background scattering effects. In some forms, the manner in which the signal is filtered may be different for different parts of the received spectrum.

In some forms, the processor 440 may apply a machine learning process to an artificial intelligence (Al) model in order to analyse the received signal. A suitable model that may be applied by the processor 440 in the analysis process may be generated using training data, for example spectra (e.g. Raman spectra) of skin lesions that are of a known type, e.g. pre-classified by a dermatologist. It will be apparent to the skilled addressee how to train a machine learning model in order to be able to classify spectra in this manner.

In certain forms, the types of skin lesions that may be represented in the training data, and consequently that may be able to be identified by the trained machine learning model, include any one or more of:
Inflammatory lesions including different eczema types (nodular prurigo, discoid eczema, atopic eczema), dermatitis, DLE (discoid lupus erythematosus), psoriasis, scabies, urticaria, hematomas, hemangiomas, rashes, morphoea, annular granuloma, epidermoid cysts, cutaneous leishmaniasis, cutaneous lupus and lichen simplex chronicus;
Cancerous lesions including melanomas (in situ, superficial spreading, spitzoid and lentiginous), basal cell carcinoas (BCC (nodular, superficial spreading) and squamous cell carcinomas (SCC); and
Neoplastic lesions including different subtypes of naevi (Becker naevi, blue naevi, compound naevi, congenital naevi, atypical naevi, halo naevi, intradermal melanocytic naevi, lentiginous junctional naevi, lentigo, linear epidermal naevi, pedunculated naevi and speckled lentiginous naevi), sebaceous hyperplasia, vascular malformation, warts, scars, sebaceomas, seborrheic keratoses, solar lentigo, cysts, actinic keratosis, keratosis ilaris, hyperkeratotic lesions, Kaposi sarcomas, fibrous papules, café au lait, angiokeratoma, acrochordon and burns.

The output of the analysis method performed by the processor 440 may be an analysis result, which in forms of the technology used to analyse a patient's skin lesion may comprise a determination of a type of skin lesion determined at the subject 500. The output may identify the type of skin lesion identified and/or a category of skin lesion, e.g. melanoma/non-melanoma, benign/malignant. In some forms, the output may comprise an indication of the confidence in the determination, e.g. a percentage risk of the skin lesion being a melanoma, or a score out of ten of the chances of the lesion being benign. That is, in some forms, the output may be an identification of a discrete quality, e.g. the type of skin lesion, or a continuous quantity, e.g. a confidence score of some characteristic. This output may amount to a diagnosis of one or more particular medical conditions, or the output may be used in the determination of such a diagnosis. Alternatively, the output may amount to an indication that no such medical condition is present.

In some forms, the training data may be pre-processed prior to being input into the Al model, for example by smoothing the training spectra, scaling the spectra to the same peak height for a reference wavelength, then mean centring the data. The pre-processed spectra may then be used to build a classification model for classifying skin lesions from their spectra. In some forms this may be achieved using a partial least squares with discriminant analysis (PLS-DA algorithm). This classification method may be more robust than algorithms like an artificial neural network (ANN), as it decomposes and reduces the data, extracting the relevant spectral information and ignoring any baseline noise while at the same time regressing the relevant features with their class information (skin lesion type, for e.g., BCC, SCC, melanoma, naevi, etc). A cross validation may also be used to validate the model, e.g. using a venetian blind method.

### 6.7. Output Device

In some forms, the analysis system 100 may comprise an output device 450 for outputting the results of the analysis performed by the processor 440. The output device 450 may be any device suitable to output information to a user and/or to another computing device, for example to another part of the analysis system 100 and/or to another system.

In some forms, the output device 450 may be physically connected to the processor 440, for example both the output device 450 and processor 440 may form part of the same computing device, e.g. PC, laptop, tablet or smartphone. In another form, the output device 450 may be physically remote from, but in communication with, the processor 440, for example able to receive information from the processor 440 via a wired or wireless communication protocol.

In some forms, the output device 450 may comprise a display screen. In other forms, the output device 450 may comprise a signal transmitter configured to output data to a remote location, e.g. over a wired or wireless communication link.

In some forms, the analysis system 100 also comprises an input device enabling a user to enter information to be provided to the analysis system 100, for example data that is provided to the processor 440. The input device may be any one or more of: a keyboard, keypad, touchpad, mouse, touchscreen display, remote communication receiver, etc. In some forms, the input device and the output device 450 are provided in the same physical device.

### 6.8. Power Supply

In certain forms of the technology, the analysis system 100 may comprise a power supply 470 configured to supply power to one or more other components in the analysis system 100. Any suitable power supply may be used, for example a battery or plurality of batteries. In some forms, the analysis system 100 may be configured to receive power from an external power source, for example a mains power supply.

### 6.9. Portable Unit

In certain forms, for example as shown in the exemplary form of FIGS. 1 and 21, the analysis system 100 may comprise a portable unit 400. Portable unit 400 may comprise one or more of the other components of the analysis system 100 and be configured to be portable to enable convenient transportation of the analysis system 100, or parts thereof, by a user.

In the form shown in FIG. 1, portable unit 400 comprises a housing 410, for example a case that is configured to be easily carried, for example the case may take the form of a box with a handle with a form similar to that of a briefcase. The case may be rigid in order to protect the components housed within it. In the form in FIG. 1, the stimulus generator 420, detector 430, processor 440 and output device 450 are housed in portable unit 400. In some forms, a power supply 470, in the form of a portable battery, may also be housed in portable unit 400. In some forms, the conduit 33 and/or probe assembly 200 may also be able to be stored in portable unit 400, and then pulled out from the portable unit 400 for use of the analysis system 100 to analyse a subject 500.

In some forms, for example as shown in FIG. 21, the analysis system 100 may comprise a probe assembly holder 402 that is configured to retain the probe assembly 200 when the probe assembly is not in use. The probe assembly holder 402 may take the form of a component, or assembly of components, forming a negative space suitable for placing the probe assembly 200, or a part of the probe assembly 200, within. In some forms, the probe assembly holder 402 may be comprised as part of portable unit 400. In the form shown in FIG. 21, the probe assembly holder 402 comprises a tube 404 that is configured (e.g. shaped and sized) to receive some or all of the housing 210 of the probe assembly 200. In the case of the housing body 10 being formed with a general form factor resembling a gun, as described above, the probe assembly holder 402 may be formed to receive the probe assembly 200 in the manner of a holster. In some forms, for example as shown, the tube 404 may have an open end 405 and a closed end 406. The probe assembly 200 may be positioned for retention in the probe assembly holder 402 by inserting the subject-proximal end of the housing 210 into the open end 405. In some forms, the probe assembly holder 402 may be configured so that the probe assembly 200 may be retained without the probe tip shield 7 being attached to the housing body 10. In other forms, the probe assembly holder 402 may be configured so that the probe assembly 200 may be retained with the probe tip shield 7 being attached to the housing body 10. In still other forms, the probe assembly holder 402 may be configured so that the probe assembly 200 may be retained with or without the probe tip shield 7 being attached to the housing body 10.

In some forms, the contents of the portable unit 400 may be considered to comprise a spectrometer.

### 6.9.1. Calibration Mechanism

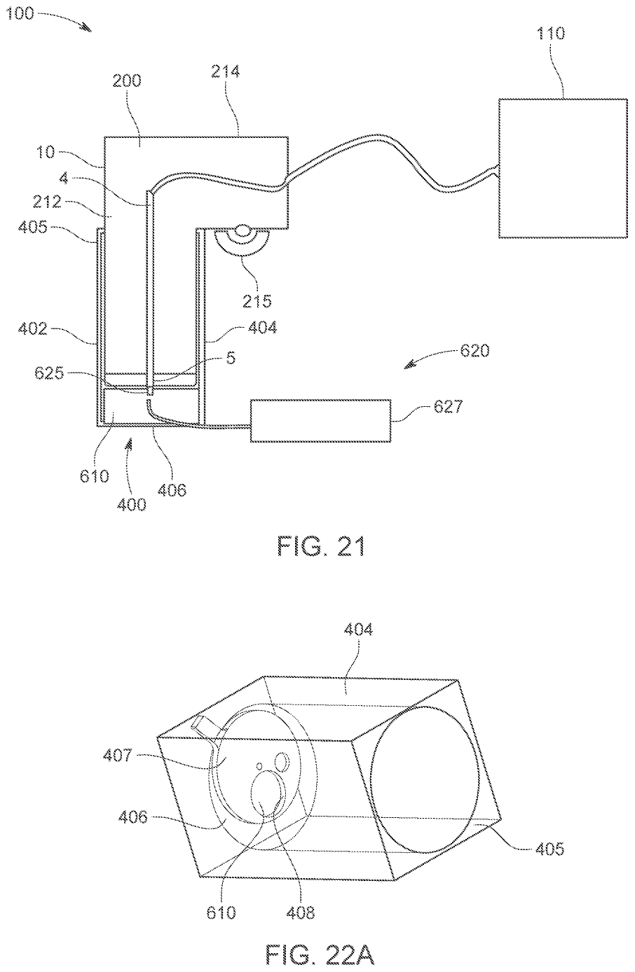

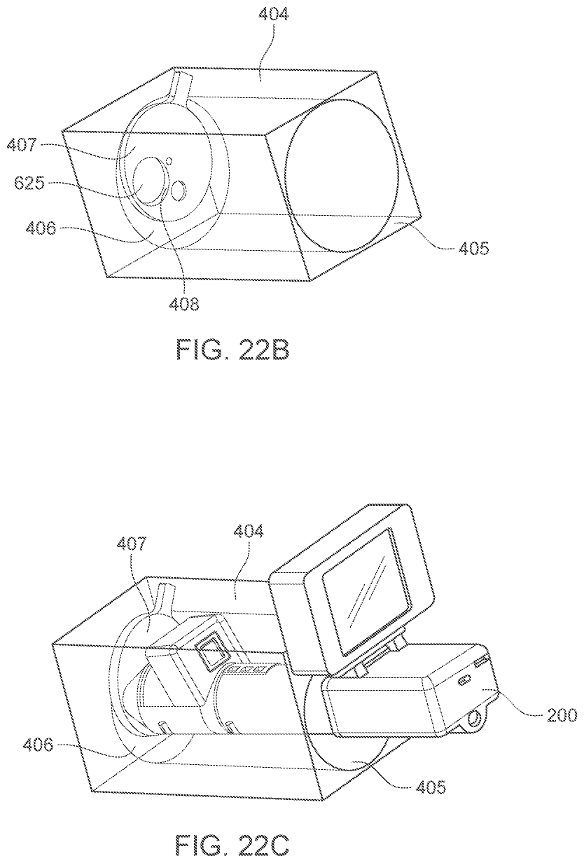

Some types of calibration mechanisms for the analysis system 100 have already been described. This section describes other types that may be used in certain forms of the technology. Examples of certain forms of calibration mechanisms are illustrated in FIGS. 21 and 22A-C.

The analysis system 100 may comprise a calibration member 610, which may be a component used to facilitate the calibration of the system, in particular calibration of the spectral analysis performed by the analysis system 100 (e.g. wavenumber calibration). The calibration member 610 may be positioned relative to the probe assembly holder 402 such that, when the probe assembly 200 is retained in the probe assembly holder 402, the calibration member 610 is positioned for analysis by the probe 4. For example, the calibration member 610 may be positioned adjacent the closed end 406 of the tube 404 so that the probe 4 is aimed at the calibration member 610 when the probe assembly 200 is retained in the probe assembly holder 402. In some forms, the calibration member 610 may be comprised as part of the probe assembly holder 402, for example the calibration member 610 may be positioned inside the tube 404 at its closed end 406. Alternatively, the calibration member 610 may be positioned outside the tube 404 but functions to close the end of the tube 404. Alternatively, the calibration member 610 may be positioned in another part of the portable unit 400 where it can easily be targeted by a user holding the probe assembly 200.

The calibration member 610 may produce a known spectrum when exposed to light from the probe 4. For example, the calibration member 610 may be formed from a material that produces a known spectrum when exposed to light of a specific wavelength, or wavelength range. In particular, in forms in which the analysis system 100 uses Raman spectroscopy, the calibration member 610 produces a known Raman spectrum when exposed to light from the probe 4. In some forms, the calibration member 1 may be formed of silicon or calcite, although other suitable materials may alternatively be used in other forms. The calibration member 610 may be formed as a monolithic lump of substance, and may have dimensions sufficiently large so that, when the probe 4 is used to analyse the calibration member 610, the detected spectrum is substantially exclusively from the calibration member 610. In some forms, the calibration member 610 may be in the form of a silicon wafer. Silicon may be a desirable substance to use to form the calibration member 610 because its Raman spectrum has a distinctive sharp peak at 521.0 cm⁻¹, which may be readily detected in the detected spectrum. Calcite also has a distinctive sharp peak (at 1086 cm⁻¹), but the intensity of this peak may vary between calcite crystals, depending on their crystal orientation and origin.

In use, the processor 440 may compare the position of a peak in the detected spectrum from the calibration member 610 with the known reference value for the peak. If these differ, the processor 440 may calibrate the analysis process accordingly, for example by introducing an offset into the signal generated by the detector 430 prior to analysing the signal, or otherwise.

Other than calibrating spectrally, the calibration mechanism may additionally, or alternatively, calibrate the power of the stimulus generator. In some forms, for example as shown in FIGS. 21 and 22A-C, the analysis system 100 may further comprise a power meter 620, which is configured to measure the power of the light emitted by the probe 4 when the stimulus generator 420 generates a stimulus. The power meter 620 may comprise a receiver 625 positioned relative to the probe assembly holder 402 such that, when the probe assembly 200 is retained in the probe assembly holder 402, the receiver 625 is positioned to receive light emitted by the probe 4. For example, the receiver 625 may be positioned adjacent the closed end 406 of the tube 404 so that the probe 4 is aimed at the receiver 625 when the probe assembly 200 is retained in the probe assembly holder 402. In some forms, the receiver 625 may be comprised as part of the probe assembly holder 402, for example the receiver 625 may be positioned inside the tube 404 at its closed end 406. Alternatively, the receiver 625 may be positioned outside the tube 404 but functions to close the end of the tube 404. Alternatively, the receiver 625 may be positioned in another part of the portable unit 400 where it can easily be targeted by a user holding the probe assembly 200. The receiver 625 may be configured to communicate with an analysis unit 627, for example through a wired or wireless connection. Dependent on the reading of the power meter 620 when the stimulus generator 420 is activated, the power of stimulus generated may be regulated to a desired level. This may mitigate against malfunction in the system causing a stimulus being generated of a power level that may be harmful to the subject 500.

In some forms, the analysis system 100 comprises both a calibration member 610 for facilitating spectral calibration of the analysis system 100 and a power meter 620 for facilitating power calibration of the analysis system 100. In such forms, the analysis system 100 may comprise a mechanism that enables both types of calibration to be readily performed. For example, there may be a calibration mechanism configured to alter the relative positions of the calibration member 610 and the receiver 625 relative to the probe assembly holder 402 such that, in a first configuration, the calibration member 610 is positioned in front of the probe 4 when the probe 4 is retained in the probe assembly holder 402 and, in a second configuration, the receiver 625 is positioned in front of the probe 4 when the probe 4 is retained in the probe assembly holder 402. It should be appreciated that, in order to alter the relative positions of the calibration member 610 and the receiver 625 relative to the probe assembly holder 402 as described, the actual position of any one or more of the calibration member 610, the receiver 625 and the probe assembly holder 402, or part thereof, may be moved in different forms of the technology. One example of such a calibration mechanism is illustrated in FIGS. 22A-C. In this form, the calibration member 610 and the receiver 625 remain in a fixed position and the mechanism is configured to move the position of a part of the probe assembly holder 402 in order to move the components between the first and second configurations. In the illustrated form, the probe assembly holder 402 comprises a tube 404 having an open end 405 and a closed end 406. In the base of the tube 404 at the closed end 406 is a movable base member 407 configured to engage with the subject-proximal end of the probe assembly 200. In the illustrated form, the base member 407 comprises an opening 408 into which the subject-proximal end of the probe assembly 200 fits. The base member 407 may be moved so that the probe assembly 200, when its subject-proximal end is fitted into the opening 408 in the base member 407, is also moved. Consequently, the probe assembly 200 may be moved between a first position, when the probe 4 is aimed at the calibration member 610, and a second position, when the probe 4 is aimed at the receiver 625. The calibration member 610 and the receiver 625 may both be positioned at the closed end 406 of the probe assembly holder 402, e.g. in the base. The opening 408 in the base member 407 may be a through-hole so that the calibration member 610 and the receiver 625 are exposed to the probe 4 when aligned with the opening 408 in the different positions of the base member 407. In the illustrated form, the base member 407 is attached to an inner surface of the closed end 406 through a pivot connection so that the base member 407 may be pivotally moved between the two positions to selectively expose either the calibration member 610 or the receiver 625 to the probe 4. In other forms, the base member 407 may be moved in a different manner, for example by sliding the base member 407 along a slide path. In some forms, the base member 407 may be moved manually, for example as illustrated in FIGS. 22A-C it may comprise a handle 409 that a user can manipulate to move the base member 407. In other forms, there may be provided a base member actuator to move the base member 407. The base member actuator may be actuated by manual control, e.g. through buttons or other controls to activate and de-activate the base member actuator. Alternatively, the base member actuator may be controlled in an automated manner. For example, when the analysis system 100 is first turned on, a calibration process may be automatically initiated. In the automated calibration process, base member actuator may first be actuated to move the base member 407 to align the probe 4 with one of the calibration member 610 and receiver 625 (if it is not already so aligned), then the stimulus generator 420 is suitably activated to be calibrated in terms of spectrum or power, as the case may be.

Subsequently, the base member actuator moves the base member 407 to align the probe 4 with the other of the calibration member 610 and receiver 625, and again the stimulus generator 420 is suitably activated for the second calibration step.

Although FIG. 9 has been described above as illustrating a part of a calibration mechanism inside the probe assembly 200, in another form, FIG. 9 may also illustrate a calibration mechanism comprised as part of a probe assembly holder 402, such as shown in FIG. 21 or 22. In this other form, what is shown in FIG. 9 may be an end view of the base of the probe assembly holder 402, with the item labelled as 1 being the calibration member and the item labelled as 2 as being the receiver. These components may be mounted so that they can be moved relative to the probe, for example rotated as shown in FIG. 9, to selectively move the calibration member and the receiver into alignment with the probe.

### 6.10. Operation of Analysis System

An example of a manner of operating an analysis system 100 in order to analyse a skin lesion on a patient according to certain forms of the technology will now be described. It will be appreciated that what is described in this section is merely one example of many ways in which an analysis system 100 may be used, but it is provided as a specific example in order to illustrate the convenience and ease of use of certain forms of the technology.

In the exemplary method, the portable unit 400 is opened and the probe assembly 200 and conduit 33 are removed from their storage cavity. The spectrometer is turned on by pushing an “on” switch, which powers up the stimulus generator 420, the detector 430, processor 440 and output device 450, the power being supplied by the power supply 470, which in this exemplary form is a rechargeable battery. Power is also supplied to the probe assembly 200, turning the probe assembly 200 on. This causes the probe assembly 200 to enter a set-up mode, which triggers the calibration process described above in order to calibrate the probe 4. The processor 440 compares the detected spectrum from the calibration member 1 with a reference spectrum stored in a memory associated with the processor 440, and determines any alteration to the spectral signals that are required as a result.

In some forms, the output device 450, which may be a display screen, prompts the user for information on the patient that is to be assessed. The user may then enter the patient information using the input device (e.g. personal information, health information, etc).

An indicator on the probe assembly 200, for example on the screen 16 of the probe assembly 200, may indicate that the probe assembly 200 is ready for use. The user then places a new, sterile assembly of a probe tip shield 7 and a skirt 6 on the end of the housing body 10. The user then places the opening at the distal end of the probe tip shield 7 over a lesion on the patient's skin, using the viewing assembly (e.g. images captured by camera 8 and displayed on screen 16) to guide positioning of the probe assembly 200. The user may then push a button in order to capture an image of the skin lesion using the camera 8. The image may be stored in a memory on the probe assembly 200 and/or in a memory associated with the processor 440, for example in the portable unit 400.

The user then pushes another button, which causes the retraction mechanism to extend the probe 4 towards the lesion (the probe 4 having been in the retracted position until now). The camera 8 may continue to capture images that are displayed on the screen 16 in order to help the user ensure that the probe 4 extends into contact with the lesion.

The user then pushes another button, which activates the spectral analysis. That is, the stimulus generator 420 is activated and light collected by the probe 4 is detected by detector 430 and analysed by processor 440. Again, the camera 8 may continue to capture images that are displayed on the screen 16 in order to help the user ensure that the probe 4 remains in contact with the desired skin lesion. This may be particularly important for a long exposure time (which in the case of Raman spectrometry may be 10 to 30 seconds).

An initial analysis may be conducted by processor 440 to determine whether a successful measurement has occurred, and may provide feedback to the user accordingly, for example by displaying an indication of ‘success’, ‘failure’ or otherwise on screen 16. Data indicative of the captured spectrum, for example raw data and/or a textual/graphical illustration of the data may be stored in a memory associated with the processor 440 and/or output on output device 450. In some forms, the memory stores the spectral data in association with image of the skin lesion captured by the camera 8. This links the spectral data and the image of the skin lesion in the memory so that a user may later view the spectral data and the image of the skin lesion to assist with identification of the lesion to which the spectral data relates.

The processor 440 may subsequently perform a more detailed analysis of the detected spectrum, and may determine an analysis result, which may include a determination of the type of skin lesion, an indication of whether the skin lesion is benign or malignant (which may be a binary result or a non-binary result, such as a percentage indication of likelihood to be malignant). The determination of the type of skin lesion may include a determination of the type of skin cancer detected, for example melanoma, non-melanoma, etc, which may be a discrete indication or continuous indication. The determined result may be output on screen 16 and/or output device 450.

### 6.11. Other Remarks

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

The technology may also be said broadly to consist in the parts, elements and features referred to or indicated in the specification of the application, individually or collectively, in any or all combinations of two or more of said parts, elements or features.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the technology and without diminishing its attendant advantages. It is therefore intended that such changes and modifications be included within the present technology.

1.-36. (canceled)

37. A probe assembly for spectral analysis of a skin region of a subject, the probe assembly comprising:
a housing configured to retain a probe for Raman spectroscopy inside the housing, wherein the housing comprises:
a housing body; and
a probe tip shield having a proximal end provided to an end of the housing body, and a distal end defining an opening to expose a tip of the probe to the skin region,
wherein the probe tip shield is configured to shield the tip from ambient light at least at wavelengths of sensitivity for Raman spectroscopy,
wherein the probe assembly is configured such that a shape of the distal end of the probe tip shield is able to be adapted to conform to a region of the subject's body surrounding the skin region, and
wherein the probe tip shield is configured such that the region of the subject's body surrounding the skin region has a minimum width sufficient to substantially eliminate ambient light scattered through a sub-surface of the subject's skin proximate the skin region from being received by the probe.

38. A probe assembly as claimed in claim 37, wherein the probe tip shield is configured to be substantially flexible such the distal end is able to conform to the region of the subject's body surrounding the skin region.

39. A probe assembly as claimed in claim 38, wherein the distal end of the probe tip shield comprises one or more cavities configured to facilitate the distal end conforming to the region of the subject's body surrounding the skin region.

40. A probe assembly as claimed in claim 38, wherein the probe tip shield comprises one or more folds enabling the probe tip shield to deform in shape to facilitate the distal end conforming to the region of the subject's body surrounding the skin region.

41. A probe assembly as claimed in claim 37, wherein the probe assembly comprises a plurality of probe tip shields, wherein the distal end of each of the plurality of probe tip shields is different in shape and/or size, and wherein the proximal end of each of the plurality of probe tip shields is interchangeably attachable to the housing body.

42. A probe assembly as claimed in claim 37, wherein the probe tip shield tapers from a wider distal end to a narrower proximal end.

43. A probe assembly as claimed in claim 37, wherein the probe tip shield is configured such that the region of the subject's body surrounding the skin region has a minimum width of approximately 5 cm.

44. A probe assembly as claimed in claim 37, wherein the probe tip shield is configured to be substantially opaque to light at substantially all wavelengths.

45. A probe assembly as claimed in claim 37, wherein the probe assembly further comprises:
a camera positioned inside the housing to capture visual images of the skin region when the distal end abuts the region of the subject's body surrounding the skin region; and
a light source to illuminate the skin region when the camera captures visual images of the skin region.

46. A probe assembly for spectral analysis of a skin region of a subject, the probe assembly comprising:
a housing configured to retain a probe for Raman spectroscopy inside the housing,
wherein the housing comprises:
a housing body; and
a probe tip shield having a proximal end provided to an end of the housing body, and a distal end defining an opening to expose a tip of the probe to the skin region, wherein the probe tip shield is configured to shield the tip from ambient light at least at wavelengths of sensitivity for Raman spectroscopy, and
wherein the probe assembly further comprises:
a camera positioned inside the housing to capture visual images of the skin region when the distal end abuts a region of the subject's body surrounding the skin region; and
a light source to illuminate the skin region with light when the camera captures visual images of the skin region.

47. A probe assembly as claimed in claim 46, wherein the probe assembly is configured such that a shape of the distal end of the probe tip shield is able to be adapted to conform to the region of the subject's body surrounding the skin region.

48. A probe assembly as claimed in claim 46, wherein the probe assembly further comprises a screen configured to display images from the camera to a user.

49. A probe assembly as claimed in claim 46, wherein the probe assembly is configured to communicate the images from the camera to a location remote from the probe assembly.

50. A probe assembly for spectral analysis of a skin region of a subject, the probe assembly comprising:
a housing configured to retain a probe for Raman spectroscopy inside the housing,
wherein the housing comprises:
a housing body; and
a probe tip shield having a proximal end provided to an end of the housing body, and a distal end defining an opening to expose a tip of the probe to the skin region,
wherein the probe tip shield is configured to shield the tip from ambient light at least at wavelengths of sensitivity for Raman spectroscopy, and
wherein the probe tip shield comprises a skirt extending radially outwardly from the distal end of the probe tip shield to cover a surface region surrounding the skin region in use, wherein the skirt is substantially opaque at least at wavelengths of sensitivity for Raman spectroscopy, and wherein a radially outer edge of the skirt has a minimum width sufficient to substantially eliminate ambient light scattered through a sub-surface of the subject's skin proximate the skin region from being received by the probe.

51. A probe assembly as claimed in claim 50, wherein the minimum width of the radially outer edge of the skirt is approximately 5 cm.

52. A probe assembly as claimed in claim 50, wherein the skirt extends radially perpendicularly outwardly from the distal end of the probe tip shield.

53. A probe assembly as claimed in claim 50, wherein the skirt is formed so as to be flexible or semi-rigid so as to be able to flex when pushed up against the subject.

54. A probe assembly as claimed in claim 50, wherein the probe assembly further comprises:
a camera positioned inside the housing to capture visual images of the skin region when the distal end abuts the region of the subject's body surrounding the skin region; and
a light source to illuminate the skin region when the camera captures visual images of the skin region.

55. A probe assembly as claimed in claim 54, wherein the probe assembly further comprises a screen configured to display images from the camera to a user.

56. A probe assembly as claimed in claim 50, wherein the probe tip shield tapers from a wider proximal end to a narrower distal end.
